(19) United States
(12) Patent Application Publication
(10) Pub. No.: US 2021/0361810 A1
(43) Pub. Date: Nov. 25, 2021

(54) MODULAR AND CONFIGURABLE SANITIZATION SYSTEM

(71) Applicant: Go Time Design, LLC, Tempe, AZ (US)

(72) Inventor: Andrew Peter Glanz, Scottsdale, AZ (US)

(73) Assignee: Go Time Design, LLC, Tempe, AZ (US)

(21) Appl. No.: 17/325,325

(22) Filed: May 20, 2021

Related U.S. Application Data
(60) Provisional application No. 63/027,432, filed on May 20, 2020.

(51) Int. Cl. A61L 2/24; A61L 2/00

(52) U.S. Cl. CPC A61L 2/24; A61L 2202/14; A61L 2/0005

(57) ABSTRACT

A modular and configurable sanitization system includes one or more panels and a processing module. The processing module is operable to obtain configuration data pertaining to one or more of: the one or more panels, one or more configurable sanitizing mechanisms of the one or more panels, and one or more sanitization subjects, determine one or more sanitization protocols based on the configuration data, generate one or more sanitizing mechanism configuration instructions to configure the one or more configurable sanitizing mechanisms based on the one or more sanitization protocols and in accordance with the one or more sanitization subjects, generate one or more panel configuration instructions to configure the one or more panels based on the one or more sanitization protocols and in accordance with the one or more sanitization subjects, and configure the one or more configurable sanitizing mechanisms and the one or more panels.

### CROSS-REFERENCE TO RELATED APPLICATIONS

The present U.S. Utility Patent application claims priority pursuant to 35 U.S.C. § 119(e) to U.S. Provisional Application No. 63/027,432, entitled “MODULAR ULTRAVIOLET (UV) LIGHT STERILIZATION SYSTEM,” filed May 20, 2020, which is hereby incorporated herein by reference in its entirety and made part of the present U.S. Utility Patent Application for all purposes.

### STATEMENT REGARDING FEDERALLY SPONSORED RESEARCH OR DEVELOPMENT

Not Applicable.

### INCORPORATION-BY-REFERENCE OF MATERIAL SUBMITTED ON A COMPACT DISC

Not Applicable.

### BACKGROUND OF THE INVENTION

### Technical Field of the Invention

This invention relates generally to sanitization mechanisms and more particularly to configurable sanitization mechanisms of a modular and configurable sanitization system.

### Description of Related Art

Ultraviolet (UV) light is a type of electromagnetic radiation falling between visible light and X-rays on the electromagnetic spectrum with wavelengths ranging from 10 nanometers (nm) to 400 nm. UV light is generally divided into three sub-bands: UVA or near UV (315-400 nm), UVB or middle UV (280-315 nm), and UVC or far UV (150-280 nm). Radiations with wavelengths from 10 nm to 180 nm are sometimes referred to as vacuum or extreme UV. These wavelengths are blocked by air and only propagate in a vacuum.

UV light is present in sunlight and has both beneficial and harmful effects on living tissue. UV photons contain enough energy to cause ionization, a process that breaks electrons away from atoms. Electron vacancies from ionization cause atoms to form chemical bonds they may not otherwise form. UVA is the majority of the UV radiation that reaches the Earth's surface and is capable of penetrating deep into living tissue, causing aging, wrinkles, sun spots, and according to some studies, contributes to sunburn and cancer. UVB is also present in sunlight and is more damaging to living tissues than UVA. UVB is the main cause of sunburn and skin cancer. However, UVB is also responsible for the formation of vitamin D in most land vertebrates including humans.

No measurable amount of UVC from solar radiation reaches the Earth's surface because ozone absorbs the shorter wavelengths. With shorter, more energetic wavelengths, UVC is effective at killing microorganisms such as bacteria and viruses by destroying the molecular bonds that hold their genetic material (DNA and/or RNA) together. Broad band UVC (e.g., from about 254 nm to 270 nm), or otherwise referred to as conventional germicidal UVC, is a staple method of sterilization used in hospitals, water treatment facilities, offices, and factories. Consumer UVC sanitization devices such as UVC phone cleaners and UVC toothbrush cleaning devices are gaining popularity. However, widespread use of conventional germicidal UVC in public settings has been limited because it is hazardous to humans. Conventional germicidal UVC is both a carcinogenic and cataractogenic. For this reason, UVC sterilization is usually done using UVC lamps with protective shields or enclosures, automatic shut-offs, and other safety features.

What is being referred to as “Far-UVC,” is a limited range of UVC having wavelengths between 207-222 nm. This limited range of UVC has been scientifically shown to inactivate bacteria and viruses without causing harm to human tissues. This limited range does not harm human (and other mammals) cells because the wavelengths are too short to penetrate human cells. Because viruses and bacteria are much smaller than human cells, far-UVC is able to penetrate those cells, damage their genetic material, and inactivate them.

Time, wavelength, distance, bulb shape, intensity, bulb wattage, type of microorganism, and distance from the target are factors is how well and fast UVC disinfects. Further, UVC only works in its light path and can be blocked by objects. As such, UVC does not necessarily clean into “cracks” (e.g., buttons of a phone, keyboard, etc.) or clean three-dimensional objects thoroughly.

Another effective sanitization method for objects and/or surfaces is application of sanitization chemicals. Sanitization chemicals have chemical properties that break down and/or destroy microbes such as bacteria. Examples of sanitization chemicals include alcohol, chlorine and chlorine compounds, formaldehyde, and hydrogen peroxide. For example, ethyl alcohol is a potent virucidal agent that inactivates all of the lipophilic viruses (e.g., influenza) and many hydrophilic viruses. The chemical properties of sanitization chemicals that break down and/or destroy microbes may also break down and/or destroy materials that are not harmful microbes (e.g., biologic tissues, equipment, surface finishes (e.g., shellac), etc.) and pollute the environment.

Air purification technology is another method of sanitization that captures and/or eliminates airborne contaminants via an air system (e.g., fan, air conditioning unit, etc.) and one or more kinds of air purification techniques. For example, a high efficiency particulate air (HEPA) filter is able to trap 99.97 percent of particles that are 0.3 microns through the use of routing air through strategically designed, maze-like, interlaced fibers. Other methods of air purification technology includes photocatalytic oxidation (PCO), activated carbon filtering, and negative ionization. PCO is a very powerful air purification technology that combines UVC irradiation with a substance (catalyst) titanium dioxide which results in a reaction that changes malignant contaminants into water, carbon dioxide, and detritus.

Water purification is another method of sanitization by which undesired chemicals, organic and inorganic materials, and biological contaminants are removed from water. Typical methods of water purification include boiling water, filtration systems (e.g., running water through an activated-carbon filter to capture and remove some water contaminants, reverse osmosis systems which uses a semipermeable membrane that block contaminants and allows water to flow through, sediment filters, etc.), sanitization chemicals (e.g., water purification liquid drops, tablets, packaged powder containing chemicals such as chlorine and iodine, etc.), water desalination technologies (e.g., reverse osmosis, vacuum distillation, multistate flash distillation, freeze-thaw, electrodialysis, etc.), and/or a combination thereof. Water treatment facilities use a combination of purification methods that begin with pretreatment (e.g., screening to remove large debris, adding sanitization pretreatment chemicals such as chlorine, adding calcium carbonate to the water, etc.), chemical treatment and refinement (e.g., adding chemicals to allow small particles to clump together in sediments, moving water through a sedimentation basin to remove sediments, etc.), filtration, and disinfection (e.g., application of ozone, UV radiation, hydrogen peroxide, chlorine, etc.).

### BRIEF DESCRIPTION OF THE SEVERAL VIEWS OF THE DRAWING(S)

FIG. 1 is a schematic block diagram of an embodiment of a modular and configurable sanitization system in accordance with the present invention;

FIG. 2 is a schematic block diagram of another embodiment of a modular and configurable sanitization system in accordance with the present invention;

FIG. 3 is a flowchart of an example of a method for configuration of a modular and configurable sanitization system in accordance with the present invention;

FIG. 4 is a schematic block diagram of another embodiment of a modular and configurable sanitization system in accordance with the present invention;

FIGS. 5A-5C are schematic block diagrams of examples of configurable sanitization mechanisms in accordance with the present invention;

FIGS. 6A-6C are schematic block diagrams of an embodiment of a modular and configurable sanitization system as a pass-through ultraviolet (UV) sanitization tunnel in accordance with the present invention;

FIGS. 7A-7B are schematic block diagrams of another embodiment of a modular and configurable sanitization system as a pass-through ultraviolet (UV) sanitization tunnel in accordance with the present invention;

FIGS. 8A-8D are schematic block diagrams of examples of pass-through UV sanitization tunnel patterns in accordance with the present invention;

FIG. 9 is a schematic block diagram of an embodiment of a modular and configurable sanitization system as a shopping cart sanitizer in accordance with the present invention;

FIG. 10 is a schematic block diagram of another embodiment of a modular and configurable sanitization system as a shopping cart sanitizer in accordance with the present invention;

FIG. 11 is a schematic block diagram of another embodiment of a modular and configurable sanitization system as a shopping cart sanitizer in accordance with the present invention;

FIG. 12 is a schematic block diagram of another embodiment of a modular and configurable sanitization system as a shopping cart sanitizer in accordance with the present invention;

FIG. 13 is a schematic block diagram of another embodiment of a modular and configurable sanitization system as a shopping cart sanitizer in accordance with the present invention;

FIG. 14 is a schematic block diagram of another embodiment of a modular and configurable sanitization system as a shopping cart sanitizer in accordance with the present invention;

FIG. 15 is a schematic block diagram of another embodiment of a modular and configurable sanitization system as a shopping cart sanitizer in accordance with the present invention;

FIG. 16 is a schematic block diagram of an embodiment of a raised platform of a modular and configurable sanitization system in accordance with the present invention;

FIG. 17 is a schematic block diagram of another embodiment of a modular and configurable sanitization system as a shopping cart sanitizing tunnel in accordance with the present invention;

FIG. 18 is a schematic block diagram of an embodiment of a modular and configurable sanitization system as a hand sanitizing station in accordance with the present invention;

FIG. 19 is a schematic block diagram of another embodiment of a modular and configurable sanitization system as a hand sanitizing station in accordance with the present invention;

FIGS. 20A-20B are schematic block diagrams of an embodiment of a modular and configurable sanitization system as a multi-purpose sanitizing device in accordance with the present invention;

FIG. 21 is a schematic block diagram of another embodiment of a modular and configurable sanitization system as a multi-purpose sanitizing device in accordance with the present invention;

FIG. 22 is a schematic block diagram of another embodiment of a modular and configurable sanitization system as a multi-purpose sanitizing device in accordance with the present invention;

FIGS. 23A-23B are a schematic block diagrams of another embodiment of a modular and configurable sanitization system as a multi-purpose sanitizing device in accordance with the present invention;

FIGS. 24A-24B are a schematic block diagrams of another embodiment of a modular and configurable sanitization system as a multi-purpose sanitizing device in accordance with the present invention; and

FIGS. 25A-25B are a schematic block diagrams of examples of sanitization subjects of a modular and configurable sanitization system as a multi-purpose sanitizing device in accordance with the present invention.

### DETAILED DESCRIPTION OF THE INVENTION

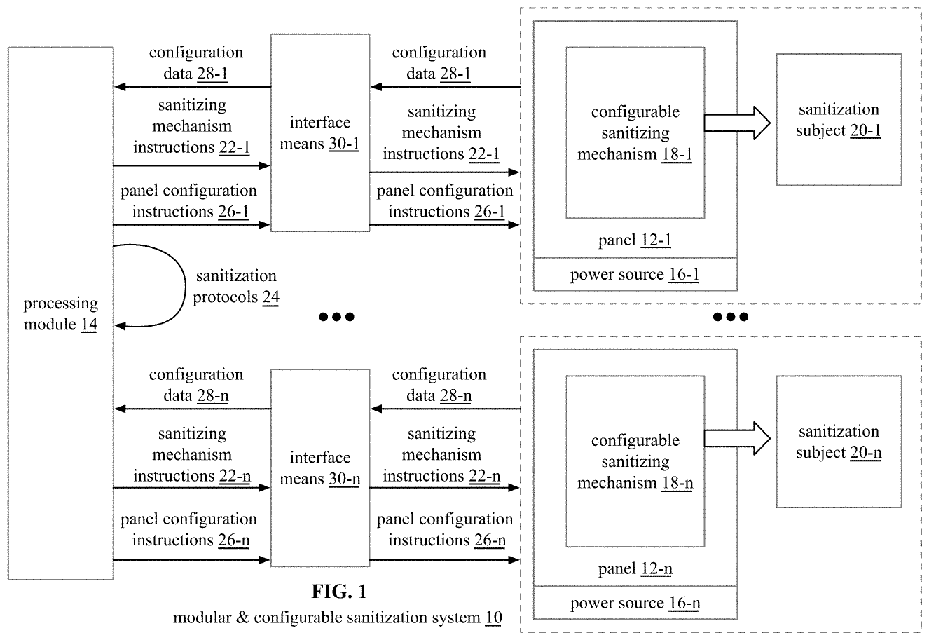

FIG. 1 is a schematic block diagram of an embodiment of a modular and configurable sanitization system 10 that includes a plurality of panels 12-1 through 12-n, a processing module 14, a plurality of power sources 16-1 through 16-n, a plurality of sanitization subjects 20-1 through 20-n, and a plurality of interface means 30-1 through 30-n. The modular and configurable sanitization system 10 is a modular system because it can be subdivided into smaller parts (i.e., the panels 12-1 through 12-n are subdividable and the sanitizing mechanisms 18-1 through 18-n are additionally subdividable) and each part can be independently created, modified, replaced, and/or exchanged with other modules or between another modular and configurable sanitization system 10.

The term “sanitization” (and likewise, “sanitize,” and “sanitizing”) is used herein interchangeably with the terms “disinfect” and “sterilize” and is intended to mean a process that substantially reduces the presence of microorganisms (e.g., bacteria, viruses, and spores) on a sanitization subject. A sanitization subject 20-1 through 20-n is any organic and/or inorganic material or portion thereof where microbes can reside such as a structural surface (e.g., a countertop, a desk, etc.), an object (e.g., a phone, food, equipment, etc.), at least a portion of a living being (e.g., a human, an animal, a human's hand, etc.), air, water, etc.

Each panel of the plurality of panels 12-1 through 12-n includes a configurable sanitizing mechanism (sanitizing mechanisms 18-1 through 18-n respectively). The panels 12-1 through 12-n are structures and/or housings made of any suitable material (e.g., metal, plastic, glass, etc.) for containing, displaying, and/or otherwise holding the configurable sanitizing mechanisms 18-1 through 18-n. The panels 12-1 through 12-n consist of an appropriate shape and size based on its respective configurable sanitizing mechanism 18-1 through 18-n, the plurality of sanitization subjects 20-1 through 20-n, functional features, a location, environmental features, aesthetics, and/or safety considerations.

The panels 12-1 through 12-n may be mechanically configurable via automatic retraction technology (e.g., a panel includes a motor, a power source, and a panel retraction housing allowing the panel to roll/unroll or fold/unfold onto the housing (e.g., a motor powered tube housing) and/or automatic collapsing technology (e.g., the panel contains a motor, a power source, and has nested sections that fit into one another to allow for an adjustable length, height, etc.) such that panels can be automatically added and/or subtracted to the system and/or the shape of at least a portion of a panel can be altered.

As another example, a material of a panel may be automatically configurable. For example, an additional panel layer may automatically unroll via retraction technology discussed above and cover a panel to alter the overall material of the panel (e.g., a tinted film is added to glass, a protective film is added based on an environmental and/or safety feature, etc.). Alternatively, a signal (e.g., from the processing module 14) may alter the chemical composition of the panel. For example, certain materials such as piezoelectric materials can bend, expand, contract, and/or alter when a signal such as voltage is applied to the material. Alternatively, a panel may contain a material that is altered by the presence and/or exposure of a physical and/or chemical property and/or condition (e.g., a panel containing a photochromic dye darkens in accordance with a level ultraviolet light exposure, etc.).

A configurable sanitizing mechanism 18-1 through 18-n is built into a respective panel, surrounded by a respective panel, in close proximity to a respective panel (e.g., attached to a fixture where the fixture is attached to the panel), embedded into a respective panel, and/or otherwise affixed to a surface of a respective panel. Examples of panel shapes, materials, and configurations are discussed in greater detail with reference to one or more of the following Figures.

The configurable sanitization mechanisms 18-1 through 18-n have configurable and/or programmable features controlled by the processing module 14. Each configurable sanitization mechanism of the configurable sanitization mechanisms 18-1 through 18-n include one or more of: one or more ultraviolet (UV) light emitting devices, one or more sanitization chemicals having one or more dispensing means, and one or more environmental purification devices (e.g., air and/or water purification devices).

The UV light emitting devices include one or more UV light-emitting diode (LED) lamps at a selectable wavelength, one or more mercury-based UV lamps operating at low vapor pressure to emit a particular wavelength (e.g., similar to fluorescent lamps), one or more pulsed-xenon UV lamps emitting UV light across the entire UVC spectrum, and/or a combination thereof. UV LED light lamps are useful for targeted sanitization applications due to selectable wavelength and more targeted, controllable light beams (e.g., traditional mercury lamps scatter light over a large area). Because certain UV light may be damaging to living tissue, far-UVC light emitting devices having wavelengths between 207-222 nm may be used when the sanitization subject 20 is a living being. Unlike conventional germicidal UVC (e.g., having wavelengths between 254 nm to 270 nm), far-UVC light inactivates bacteria and viruses without harming human tissue.

UV light lamp type and wattage is selectable based on the distance to a sanitization subject and desired dose of UV necessary for sanitization. For example, UV dose is generally calculated at 1 meter from a lamp. To ensure effectiveness, the UV dose is calculated based on UV intensity, distance to target, bulb and/or panel coating (e.g., shatter proof, scratch resistant, etc., can reduce UV output), end of lamp life, airflow, and exposure time. UV intensity is increased by introducing multiple UV lamps. The panels 12-1 through 12-n may include light filtering capabilities via different materials, lenses, etc., in order to affect the intensity, frequency, and/or wavelength of the beams in order to produce a desired effect (e.g., specific intensities and wavelengths selected depending on the target, etc.).

Examples of sanitization chemicals include alcohol, chlorine and chlorine compounds, formaldehyde, and hydrogen peroxide. For example, ethyl alcohol is a potent virucidal agent that inactivates all of the lipophilic viruses (e.g., influenza) and many hydrophilic viruses. As a specific example, alcohol-based hand sanitizers may be a liquid, gel, or foam containing 60-95% alcohol by volume, additional antiseptics such as chlorhexidine and quaternary ammonium derivatives, sporicides such as hydrogen peroxide, emollients and gelling agents to reduce skin dryness and irritation, a small amount of distilled or sterile water, and sometimes foaming agents, colorants, and/or fragrances.

Depending on the sanitization subject 20, the type and/or composition of sanitization chemicals desired and/or required, and/or the state and/or format of the sanitization chemicals (e.g., liquid, foam, etc.), there are a variety of dispensing means for sanitization chemicals. For example, a liquid, foam, or lotion alcohol-based hand sanitizer can be dispensed via an automatic pump (e.g., triggered by a proximity sensor, an infrared sensor, etc.). As another example, a liquid, foam, or lotion alcohol-based hand sanitizer may be contained and/or applied to an item (e.g., inserted into a dissolvable casing, applied to a tissue, sponge, cloth, etc.) where the item is dispensed via an appropriate mechanism (e.g., an automatic paper towel dispenser triggered by motion sensing, etc.) or dispenses a sanitization chemical upon touch (e.g., a sponge applicator of a sanitization chemical is affixed to a door knob (and/or handle) and the sanitization chemical is dispensed when a user touches the door knob).

An air purification device captures and/or eliminates airborne contaminants by sucking in and circulating air (e.g., via an air system such as a fan, air conditioning unit, etc.) through one or more air purification techniques. For example, a high efficiency particulate air (HEPA) filter is an air purification technique that traps 99.97 percent of particles that are 0.3 microns through the use of routing air through strategically designed, maze-like, interlaced fibers. Other air purification techniques include photocatalytic oxidation (PCO), activated carbon filtering, and negative ionization. PCO is an air purification technique that combines UVC irradiation with a substance (i.e., a catalyst) titanium dioxide which results in a reaction that changes malignant contaminants into water, carbon dioxide, and detritus.

A water purification device runs water through one or more water purification techniques such as carbon filtering, reverse osmosis, sanitization chemicals, ozone, and/or UV light for disinfection, water desalination techniques, and/or a combination thereof. In another embodiment, the water purification device includes a dispensing means for dispensing water sanitization chemicals to a user (e.g., a device that dispenses water purification tablets).

The plurality of power sources 16-1 through 16-n may be voltage supply circuits (e.g., a battery, a linear regulator, an unregulated DC-to-DC converter, etc.) to produce a voltage-based power signal, a current supply circuit (e.g., a current source circuit, a current mirror circuit, etc.) to produce a current-based power signal, or a circuit that provide a desired power level to the one or more panels 12-1 through 12-n. As shown, the power sources 16-1 through 16-n are included in the panels 12-1 through 12-n respectively (e.g., a battery power source).

Alternatively, the one or more of the power sources 16-1 through 16-n are external power sources where one or more of the panels 12-1 through 12-n includes a wired and/or wireless connection to a respective power source. In another embodiment, one or more of the panels 12-1 through 12-n derive power from one or more shared power sources of the power sources 16-1 through 16-n. In another embodiment, one or more of the power sources 16-1 through 16-n is included in the processing module 14.

The processing module 14 is described in greater detail at the end of the detailed description of the invention section and is operable to communicate with one or more of the panels 12-1 through 12-n, one or more of the power sources 16-1 through 16-n, and/or one or more computing devices associated with one or more of the sanitization subjects 20-1 through 20-n (e.g., a sanitization subject of the sanitization subjects 20-1 through 20-n is one or more computing devices, a sanitization subject of the sanitization subjects 20-1 through 20-n is a user of the one or more computing devices, etc.) via one or more of the plurality of interface means 30-1 through 30-n.

An interface means of the interface means 30-1 through 30-n includes one or more of: near-field communication (NFC), radio frequency identification (RFID) technology, and/or any wired and/or wireless communication connection. A wired communication connection includes a Gigabit LAN connection, a Firewire connection, and/or a proprietary computer wired connection. A wireless communication connection includes a wireless local area network (WLAN) communication connection, a cellular communication connection, a Bluetooth communication connection, and/or a ZigBee communication connection.

The processing module 14 may be a standalone computing device, operating on one or more standalone computing devices, and/or included in one or more of: one or more of the power sources 16-1 through 16-n, one or more computing devices associated with the modular and configurable sanitization system 10, and/or one or more of the panels 12-1 through 12-n. The one or more computing devices associated with the modular and configurable sanitization system 10 include one or more computing devices associated with one or more of the sanitization subjects 20-1 and 20-n.

For example, a sanitization subject is a human being operating a user computing device where the user computing device is operable to communicate with (and/or includes) the processing module 14 regarding a desired, preferred, and/or required sanitization process(es). As another example, the sanitization subject is a user computing device (e.g., a smart phone) where the user computing device is operable to communicate with (and/or includes) the processing module 14 regarding a desired, preferred, and/or required sanitization process(es). As another example, a system administrator operates a user computing device where the user computing device is operable to communicate with (and/or includes) the processing module 14 regarding a desired, preferred, and/or required sanitization process(es).

The one or more computing devices associated with the modular and configurable sanitization system 10 may be fixed and/or portable computing devices. The processing module 14 may be a processing module of a fixed and/or portable computing device. A portable computing device may be a social networking device, a gaming device, a cell phone, a smart phone, a digital assistant, a digital music player, a digital video player, a laptop computer, a handheld computer, a tablet, a video game controller, and/or any other portable device that includes a computing core. A fixed computing device may be a computer (PC), a computer server, a cable set-top box, a satellite receiver, a television set, a printer, a fax machine, home entertainment equipment, a video game console, and/or any type of home or office computing equipment.

In an embodiment, the processing module 14 includes a data analysis module having machine learning and/or artificial intelligence functionality to automate features of the modular and configurable sanitization system 10 based off of an analytical model that learns from data of the modular and configurable sanitization system 10, identifies patterns of the modular and configurable sanitization system 10, and is operable to automatically make decisions regarding the modular and configurable sanitization system 10 without human interaction.

The processing module 14 is operable to obtain configuration data 28-1 through 28-n regarding one or more of the panels 12-1 through 12-n, one or more of the power sources 16-1 through 16-n, one or more of the configurable sanitizing mechanisms 18-1 through 18-n, one or more sanitization subjects 20-1 through 20-n, and/or one or more computing devices associated with the modular and configurable sanitization system 10.

The processing module 14 obtains the configuration data 28-1 through 28-n via one or more of: user input data (e.g., via a user associated one or more computing devices associated with the modular and configurable sanitization system 10), a query and response process, (e.g., with the panels 12-1 through 12-n and/or the power sources 16-1 through 16-n via one or more interface means 30-1 through 30-n), an analysis of historical and/or current data, and/or a lookup (e.g., via local memory of one or more of the processing module 14, one or more of the panels 12-1 through 12-n, one or more of the power sources 16-1 through 16-n, and/or one or more computing devices associated with the modular and configurable sanitization system 10; via a database; via cloud storage, etc.).

While the configuration data 28-1 through 28-n is shown in this example as coming from one or more of: the panels 12-1 through 12-n, the power sources 16-1 through 16-n, the configurable sanitizing mechanisms 18-1 through 18-n, and/or the sanitization subjects 20-1 through 20-n, the processing module 14 may obtain the data locally as discussed above (e.g., from local memory, via generation), and/or from external sources (e.g., a user computing device, a system administrator computing device, cloud storage, database etc.).

The processing module 14 obtains the configuration data 28-1 through 28-n during a calibration and/or set up phase, at pre-determined time intervals, dynamically (e.g., as user input is received, as system issues are detected, etc.), upon power-up (e.g., a power up of one or more power sources 16-1 through 16-n and/or the processing module 14), and/or upon request). A system issue includes one or more of: detection of an expiration time period (e.g., a panel and/or a configurable sanitization mechanism is past a warranty and/or expiration date, etc.), detection of a safety and/or security issue (e.g., a sanitization mechanism is broken, faulty, consuming too much or too little power, a sanitization subject is too close to a sanitization mechanism, a sanitization subject is using a sanitization mechanism incorrectly, etc.), detection of an unauthorized use (e.g., tampering of a sanitization mechanism is detected), a connectivity issue (e.g., a network connection is lost).

The configuration data 28-1 through 28-n includes one or more of: characteristics of the sanitization subjects 20-1 through 20-n (e.g., an identification (ID) of a sanitization subject, a type of a sanitization subject, dimensions of a sanitization subject, a material of a sanitization subject, etc.), sanitization requirements of one or more of the sanitization subjects 20-1 through 20-n (e.g., a sanitization method requirement, a sanitization dosage requirement, a required time period of sanitization, etc.), sanitization preferences of one or more of the sanitization subjects 20-1 through 20-n (e.g., a sanitization method preference, a sanitization dosage preference, a preferred time period of sanitization, etc.), default settings regarding one or more of the sanitization subjects 20-1 through 20-n (e.g., a default sanitization method, a default sanitization dosage, a default time period of sanitization, etc.), a required panel configuration of one or more of the panels 12-1 through 12-n, a desired panel configuration of one or more of the panels 12-1 through 12-n, a current panel configuration of one or more of the panels 12-1 through 12-n, a default panel configuration of one or more of the panels 12-1 through 12-n, characteristics of configurable sanitizing mechanism of one or more of the configurable sanitizing mechanisms 18-1 through 18-n (e.g., a type of configurable sanitizing mechanism, configurable sanitizing mechanisms capabilities, configurable sanitizing mechanisms strength, configurable sanitizing mechanisms usage instructions, etc.), configuration capabilities of one or more of the configurable sanitizing mechanisms 18-1 through 18-n (e.g., a combination of configurable sanitizing mechanisms is possible, dosage settings are configurable, etc.), default settings of the configurable sanitizing mechanisms 18-1 through 18-n (e.g., a default UV bulb wattage, a default sanitization chemical dose, etc.), and power source information of the one or more power sources 16-1 through 16-n (e.g., power source health, power source requirements, power source default settings, power availability, etc.).

For example, the configuration data 28-1 through 28-n indicates that the configurable sanitizing mechanisms 18-1 through 18-n each contain a plurality of UV light-emitting diode (LED) lamps having selectable wavelengths and/or wattages, the sanitization subjects 20-1 through 20-n are shopping carts of a certain height, width, and length, and the current panel configuration is a shopping cart tunnel configuration. As another example, the configuration data 28-1 through 28-n indicates that one or more of the configurable sanitizing mechanisms 18-1 through 18-n contains sanitization chemicals having one or more sanitization chemical dispensing means and the configuration data 28-1 through 28-n indicates a need and/or desire for a particular sanitization chemical dispensing mechanism. As another example, the configuration data 28-1 through 28-n indicates a combination of UV LED lamps and sanitization chemicals having one or more sanitization chemical dispensing means (e.g., a shopping cart sanitization tunnel with automatic hand sanitizing dispensing mechanisms affixed to the exterior of the tunnel).

Based on the configuration data 28-1 through 28-n, the processing module 14 determines a plurality of sanitization protocols 24 regarding configuration of the modular and configurable sanitization system. The plurality of sanitization protocols 24 include one or more of: one or more methods of sanitization (e.g., type of sanitization mechanism that should be used and the means of application), an amount (i.e., dosage) of sanitization, a time period of sanitization, safety protocols of the sanitization, and an area of sanitization (e.g., the size and shape of structure needed to accommodate sanitizing the sanitization subject). Determining the plurality of sanitization protocols 24 involves analyzing at least a portion of the configuration data 28-1 through 28-n and balancing desired features, requirements, feasibility, and safety of the at least a portion of the configuration data 28-1 through 28-n to determine the most appropriate sanitization protocols.

For example, continuing the shopping cart example from above, the processing module 14, determines sanitization protocols 24 pertaining to shopping cart sanitization in accordance with the configuration data 28-1 through 28-n that. For example, the processing module 14 performs a lookup of default procedures regarding sanitization protocols for cart sanitization and determines a desired and safe wattage for the UV lamps available. Alternatively or additionally, the processing module 14 generates the sanitization protocols 24 by analyzing the configuration data 28-1 through 28-n and/or using a sanitization protocol data model (e.g., instead of performing a lookup of safe and effective wattages for shopping cart sanitization, a data analysis module with machine learning functionality determines a safe and effective wattage based on historical data and an analytic model of the desired system).

Continuing the example, the processing module 14 may determine that the sanitization protocols 24 for the shopping cart include using a particular UV lamp type having a particular UV wavelength and/or wattage based on the amount of shopping carts involved, the structure of the tunnel and/or device for effective sanitization and safety, the characteristics of the UV LED lamps (e.g., bulb and/or panel coating (e.g., shatter proof, scratch resistant, etc.), end of lamp life, etc.), and the power needed to produce a desired dose of UV required for sanitization of the shopping carts. The sanitization protocols 24 may further include a set of safety protocols (e.g., a maximum UV dosage and exposure time, an automatic shut off function, a pattern of UV wavelength and/or wattages, etc.). The sanitization protocols 24 may further include a protocol to re-configure the shopping cart tunnel panels by adding additional panels, removing panels, changing the shape of panels, and/or altering the material of the panels via automatic means.

As another example (continuing the sanitization chemical example from above) the processing module 14 generates sanitization protocols 24 regarding a particular dosage of sanitization chemicals to be dispensed per use (e.g., based on the sanitization subject and the type of sanitization chemical) and/or a selection of sanitization chemicals and/or dispensing means when more than one option is available. The sanitization protocols 24 may further include a protocol to re-configure the panels housing the sanitization chemicals by adding additional panels, removing panels, changing the shape of panels, and/or altering the material of the panels.

Based on the plurality of sanitization protocols 24, the processing module 14 generates one or more control signals to the panels 12-1 through 12-n and/or the power sources 16-1 and 16-n via the interface means 30-1 through 30-n. The control signals include sanitizing mechanism instructions 22-1 through 22-n and panel configuration instructions 26-1 through 26-n. The processing module 14 configures the one or more configurable sanitizing mechanisms and the one or more panels based on the one or more sanitizing mechanism configuration instructions and the one or more panel configuration instructions by sending the control signals to various components or by enacting the configuration directly.

The sanitizing mechanism instructions 22-1 through 22-n include instructions on how to configure the configurable sanitizing mechanisms 18-1 through 18-2 based on the sanitization protocols 24. For example, the configurable sanitizing mechanisms 18-1 through 18-2 may include instructions to activate and/or inactivate one of the configurable sanitizing mechanisms 18-1 through 18-2.

For example, continuing the shopping cart example from above, the sanitizing mechanism instructions 22-1 through 22-n includes instructions to set wavelengths and/or wattages of each UV LED lamp. The sanitizing mechanism instructions 22-1 through 22-n may further include instructions to activate and/or inactivate one or more UV LED lamps based on the desired and/or required dose of UV (e.g., UV intensity is increased by introducing multiple UV lamps).

The sanitizing mechanism instructions 22-1 through 22-n may further include instructions to set of wavelengths and/or wattages for activating and/or inactivating one or more UV LED lamps in accordance with a pattern set forth by the sanitization protocols 24. For example, a higher UV wavelength may be used in the center of the shopping cart tunnel and lower UV wavelengths may be used towards the entrance and exit of the tunnel where human presence is more likely. The sanitizing mechanism instructions 22-1 through 22-n may further include instructions to initiate a safety protocol where when a door to the shopping cart tunnel (e.g., an entrance and/or exit door) is opened, power to the UV lamps shut off or the wattage and/or wavelength of the UV lamps is reduced to a UV wavelength that is safe for human contact.

As another example, continuing the sanitization chemical example, the sanitizing mechanism instructions 22-1 through 22-n include instructions for activating the sanitization chemical dispensing means, instructions indicating the particular dosage of sanitization chemicals to be dispensed per use, and/or instructions to select particular sanitization chemicals and/or dispensing means when more than one option is available.

The panel configuration instructions 26-1 through 26-n include instructions on how to configure the panels 12-1 through 12-n based on the sanitization protocols 24. For example, the panel configuration instructions 26-1 through 26-n may include instructions to remove and/or add a panel in accordance with the sanitization protocols 24. As another example, the panel configuration instructions 26-1 through 26-n may include an instructions to alter the shape of a panel in accordance with a desired panel configuration. As another example, the panel configuration instructions 26-1 through 26-n may include an instructions to alter the material of a panel in accordance with a desired panel configuration.

The processing module 14 may additionally send control signals to one or more of the power sources 16-1 through 16-n regarding power changes and/or requirements based on the sanitization protocols 24. When a sanitization subject is associated with a user computing device, the processing module may additionally send signals to the user computing device and/or system display regarding the sanitization protocols 24 (e.g., notifications, messages, instructions on how to use the modular and configurable sanitization system, warnings and precautions, etc.).

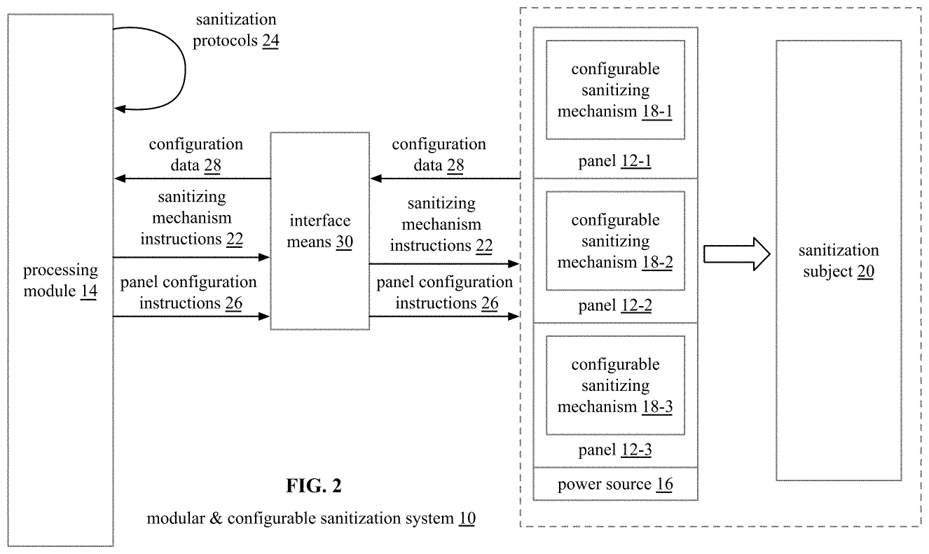

FIG. 2 is a schematic block diagram of another embodiment of a modular and configurable sanitization system 10 that includes a plurality of panels 12-1 through 12-3, a processing module 14, a power source 16, a sanitization subject 20, and an interface means 30. The modular and configurable sanitization system 10 of FIG. 2 operates similarly to the modular and configurable sanitization system 10 of FIG. 1 except that FIG. 2 depicts an example where panels 12-1 through 12-3 form a panel array powered by a shared power source 16 to sanitize a particular sanitization subject 20.

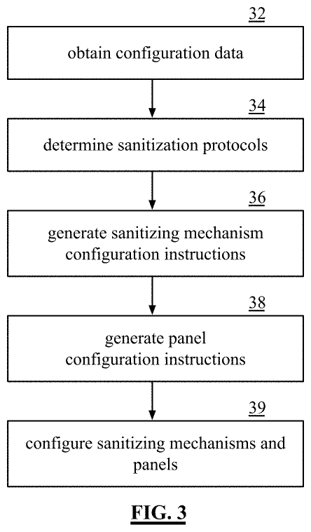

FIG. 3 is a flowchart of an example of a method for configuration of a modular and configurable sanitization system. The method begins with step 32 where a processing module of the modular and configurable sanitization system obtains configuration data of the modular and configurable sanitization system (e.g., during a calibration and/or set up phase, at pre-determined time intervals, dynamically (e.g., as user input is received, as system issues are detected, etc.), upon power-up (e.g., of one or more of the power sources and/or the processing module), and/or upon request).

A system issue includes one or more of: detection of an expiration time period (e.g., a panel and/or a configurable sanitization mechanism is past a warranty and/or expiration date, etc.), detection of a safety and/or security issue (e.g., a sanitization mechanism is broken, faulty, consuming too much or too little power, a sanitization subject is too close to a sanitization mechanism, a sanitization subject is using a sanitization mechanism incorrectly, etc.), detection of an unauthorized use (e.g., tampering of a sanitization mechanism is detected), a connectivity issue (e.g., a network connection is lost).

The configuration data includes one or more of: characteristics of one or more sanitization subjects of the modular and configurable sanitization system (e.g., an identification (ID) of a sanitization subject, a type of a sanitization subject, dimensions of a sanitization subject, a material of a sanitization subject, etc.), sanitization requirements of one or more of the sanitization subjects (e.g., a sanitization method requirement, a sanitization dosage requirement, a required time period of sanitization, etc.), sanitization preferences of one or more of the sanitization subjects (e.g., a sanitization method preference, a sanitization dosage preference, a preferred time period of sanitization, etc.), default settings regarding one or more of the sanitization subjects (e.g., a default sanitization method, a default sanitization dosage, a default time period of sanitization, etc.), a required panel configuration of one or more of panels of the modular and configurable sanitization system, a desired panel configuration of one or more of the panels, a current panel configuration of one or more of the panels, a default panel configuration of one or more of the panels, characteristics of configurable sanitizing mechanism of one or more of the configurable sanitizing mechanisms, configuration capabilities of one or more of the configurable sanitizing mechanisms, default settings of the configurable sanitizing mechanisms, and power source information of the one or more power sources (e.g., power source health, power source requirements, power source default settings, power availability, etc.).

The method continues with step 34 where, based on the configuration data, the processing module determines a plurality of sanitization protocols regarding configuration of the modular and configurable sanitization system. The plurality of sanitization protocols include one or more of: one or more methods of sanitization, an amount (i.e., dosage) of sanitization, a time period of sanitization, safety protocols of the sanitization, and protocols regarding the area to be sanitized. For example, the sanitization protocols may include a protocol to re-configure the panels housing the sanitization chemicals by adding additional panels, removing panels, changing the shape of panels, and/or altering the material of the panels based off of configuration data indicated a change in a sanitization subject, safety update, etc. Determining the plurality of sanitization protocols involves analyzing at least a portion of the configuration data to balance desired features, requirements, feasibility, and safety.

The method continues with step 36 where, based on the plurality of sanitization protocols, the processing module generates one or more sanitizing mechanism instructions to configure the one or more sanitizing mechanisms. The sanitizing mechanism instructions include instructions on how to configure the configurable sanitizing mechanisms based on the sanitization protocols. For example, the configurable sanitizing mechanisms may include instructions to activate and/or inactivate one of the configurable sanitizing mechanisms (e.g., a control signal is sent to the power source to turn on a UV lamp at a particular wattage).

The method continues with step 38, where based on the plurality of sanitization protocols, the processing module generates panel configuration instructions to configure the one or more panels. For example, the panel configuration instructions may include instructions to remove and/or add a panel in accordance with the sanitization protocols. As another example, the panel configuration instructions may include an instructions to alter the shape of a panel in accordance with a desired panel configuration (e.g., via signals to a retraction motor device of one or more panels). As another example, the panel configuration instructions may include an instructions to alter the material of a panel in accordance with a desired panel configuration (e.g., via a signal to a power source to generate a particular voltage across a panel to change its material properties).

The method continues with step 40 where the processing module configures the one or more configurable sanitizing mechanisms and the one or more panels based on the one or more sanitizing mechanism configuration instructions and the one or more panel configuration instructions by sending the control signals to various components or by enacting the configuration directly.

For example, the processing module sends the sanitizing mechanism instructions and/or the panel configuration instructions to one or more panels, the one or more power sources, and/or the one or more sanitization subjects of the of the modular and configurable sanitization system to configure to one or more panels, the one or more power sources, and/or the one or more sanitization subjects of the of the modular and configurable sanitization system to configure.

The processing module may additionally send control signals to one or more of the power sources regarding power changes and/or requirements based on the sanitization protocols. When a sanitization subject is associated with a user computing device, the processing module may additionally send signals to the user computing device regarding the sanitization protocols (e.g., notifications, messages, instructions on how to use the modular and configurable sanitization system, warnings and precautions, etc.).

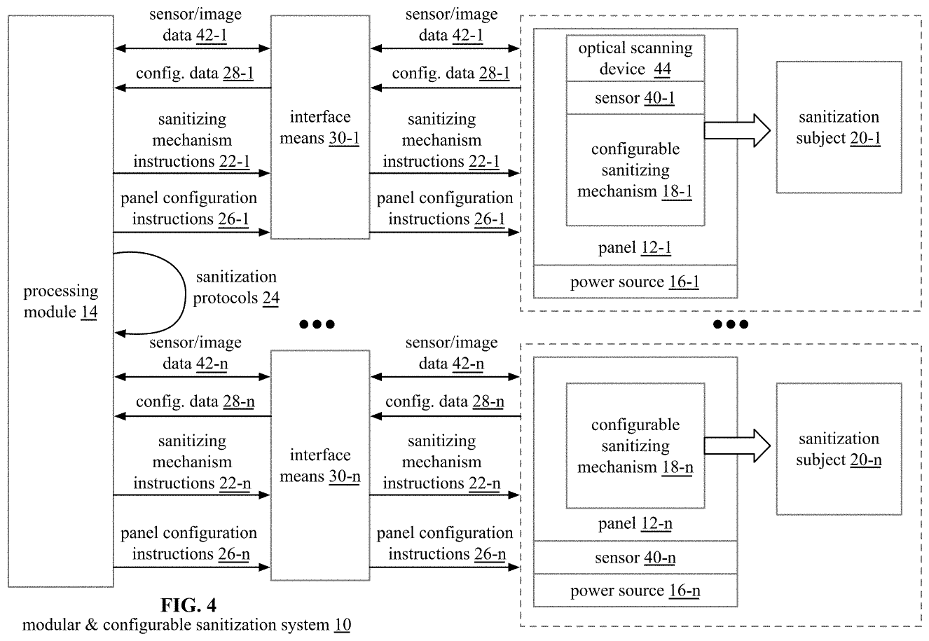

FIG. 4 is a schematic block diagram of another embodiment of a modular and configurable sanitization system 10 that includes a plurality of panels 12-1 through 12-n, a processing module 14, a plurality of power sources 16-1 through 16-n, a plurality of sanitization subjects 20-1 through 20-n, a plurality of interface means 30-1 through 30-n, and a plurality of sensors 40-1 through 40-n. The plurality of panels 12-1 through 12-n include a plurality of configurable sanitizing mechanisms 18-1 through 18-n. The modular and configurable sanitization system 10 of FIG. 4 operates similarly to the modular and configurable sanitization system 10 of FIG. 1 except that FIG. 4 includes sensor technology and/or optical scanning technology incorporated into one or more of the plurality of panels 12-1 through 12-n, the power sources 16-1 through 16-n, and/or the sanitization subjects 20-1 through 20-n.

For example, as shown in FIG. 4, the panel 12-1 includes a configurable sanitizing mechanism 18-1 where the configurable sanitizing mechanism 18-1 is coupled to a sensor 40-1 and an optical scanning device 44. The panel 12-n is coupled to a sensor 40-n. The panels 12-1 through 12-n and/or configurable sanitizing mechanisms 18-1 through 18-n may include more or less sensors and/or optical scanning devices 44. For example, the panel 12-1 may include a plurality of sensors. As another example, the panels 12-1 through 12-n are coupled to one another and to at least one sensor 40 and at least one optical scanning device 44.

The optical scanning device 44 is operable to capture and/or interpret image data via video, camera, infrared (IR) scanning, barcode scanning, and/or any type of optical scanning technology. The sensors 40-1 through 40-n function to convert a physical input into an electrical output and/or an optical output. The physical input of a sensor may be one of a variety of physical input conditions. For example, the physical condition includes one or more of, but is not limited to, acoustic waves (e.g., amplitude, phase, polarization, spectrum, and/or wave velocity); a biological and/or chemical condition (e.g., fluid concentration, level, composition, etc.); an electric condition (e.g., charge, voltage, current, conductivity, permittivity, eclectic field, which includes amplitude, phase, and/or polarization); a magnetic condition (e.g., flux, permeability, magnetic field, which amplitude, phase, and/or polarization); an optical condition (e.g., refractive index, reflectivity, absorption, etc.); a thermal condition (e.g., temperature, flux, specific heat, thermal conductivity, etc.); and a mechanical condition (e.g., position, velocity, acceleration, force, strain, stress, pressure, torque, etc.). For example, piezoelectric sensor converts force or pressure into an eclectic signal. As another example, a microphone converts audible acoustic waves into electrical signals.

There are a variety of types of sensors to sense the various types of physical conditions. Sensor types include, but are not limited to, capacitor sensors, inductive sensors, accelerometers, piezoelectric sensors, light sensors, magnetic field sensors, ultrasonic sensors, temperature sensors, infrared (IR) sensors, touch sensors, proximity sensors, pressure sensors, level sensors, smoke sensors, and gas sensors.

The processing module 14 is operable to send and receive sensor/image data 42-1 through 42-n via the interface means 30-1 through 30-n. The sensor/image data 42-1 through 42-n includes sensor/image signals sent by the processing module 14, sensor/image information obtained by the sensors 40-1 through 40-n, and/or information obtained and/or interpreted by the optical scanning device 44. Sensor/image signals sent by the processing module 14 may include signals to turn on or off sensors and/or the optical scanning device 44, to adjust a type of sensing, to adjust a strength of sensing, to adjust a frequency of sensing, etc.

The sensor information obtained by the sensors 40-1 through 40-n depends on the type of sensors used and/or the sanitization subjects 20-1 through 20-n of the modular and configurable sanitization system 10. For example, a proximity sensor detects when a sanitization subject 20-1 through 20-n is present and send sanitization subject 20-1 through 20-n presence information to the processing module 14. When an intended sanitization subject 20-1 through 20-n is of a certain size, shape, and/or material, the proximity sensor can be set to trigger only when an intended sanitization subject 20-1 through 20-n is present and to ignore unintended subjects (e.g., subjects that are too small, that are moving too fast, etc.).

Similarly, a touch and/or pressure sensor is operable to detect when a sanitization subject 20-1 through 20-n is present based off of a sanitization subject 20-1 through 20-n's touch or applied pressure on the sensor and is operable to send sanitization subject 20-1 through 20-n presence information to the processing module 14 and to ignore unintended subjects (e.g., unintended touches (e.g., an elbow compared to a finger touch), subjects that do not apply enough pressure (e.g., a bag on a pressure sensor seat does not trigger a presence whereas a human being does.), etc.).

The optical scanning device 44 is operable to send sanitization subject 20-1 through 20-n presence information based off of captured image data of the sanitization subjects 20-1 through 20-n (e.g., photographic images, video data, barcode scan to identify an individual and/or item, etc.) as the sensor/image data 42-1 through 42-n to the processing module 14.

The sensor/image data 42-1 through 42-n obtained by the sensors 40-1 through 40-1 and/or the optical scanning device 44 can inform the processing module 14 to apply a particular sanitization method, to dispense a particular sanitization dose (e.g., an amount of sanitization chemicals, and amount of UV light, etc.), to turn off power to one or more components of the modular and configurable sanitization system, etc. The sensor/image data 42-1 through 42-n may be included in the configuration data 28-1 through 28-n such that the processing module 14 uses the sensor/image data 42-1 through 42-n to determine one or more of the sanitization protocols 24.

As a specific example, UV light only disinfects what is directly in its light path. Sensing and tracking technology may be used to better direct light onto the irregular contours (e.g., such as an individual's body). For example, a proximity sensor detects a sanitization subject is present, infrared sensing and/or optical scanning technology (e.g., camera, etc.) detects the shape, size, rate of movement, etc. of the sanitization subject. The proximity, shape, and tracking data is sent as sensor/image data 42-1 through 42-n to the processing module 14 for analysis. For example, processing module 14 produces a sanitization protocol to adjust UV bulbs to direct light at an object in appropriate angles at certain times based off of the sensor/image data 42-1 through 42-n.

Other technologies such as beam manipulation and light direction, may be incorporated with sensor and/or image capture technology for various applications. For example, a panel embedded with UV light bulbs may include scratch and/or dust sensing technology such as infrared sensing to detect scratches and/or dust on the panel surface that could affect the amount of UV light emitted. When a scratch and/or or dust is detected, the processing module is operable to produce a sanitization protocol to send signals to the UV light bulbs to adjust the UV light to compensate for the scratch and/or dust. For example, the UV light bulb arrays are equipped with beam manipulation and light direction technology controlled by the processing module to direct light around a blocked area.

As another example, the processing module produces a sanitization protocol to turn off bulbs affected by dust or scratches to save power. As another example, the processing module produces a sanitization protocol to send a message to a user computing device associated with a user of the modular and configurable sanitization system notifying the user that cleaning and/or repair is necessary for proper function. As another example, the modular and configurable sanitization system includes a display (e.g., a touchscreen, an LED display, a liquid crystal display (LCD) flat panel, etc.) and the processing module produces a sanitization protocol to display a message on the display notifying a viewer of the display that cleaning and/or repair is necessary for proper function.

Alternatively or in addition to automatic bulb adjustment, light filtering and beam direction can be used to direct beams of UV light to desired areas. Light filtering through different materials, lenses, etc., can also be used to adjust the intensity, frequency, and/or wavelength of the beams in order to produce a desired effect (e.g., specific intensities and wavelengths selected depending on the target, etc.).

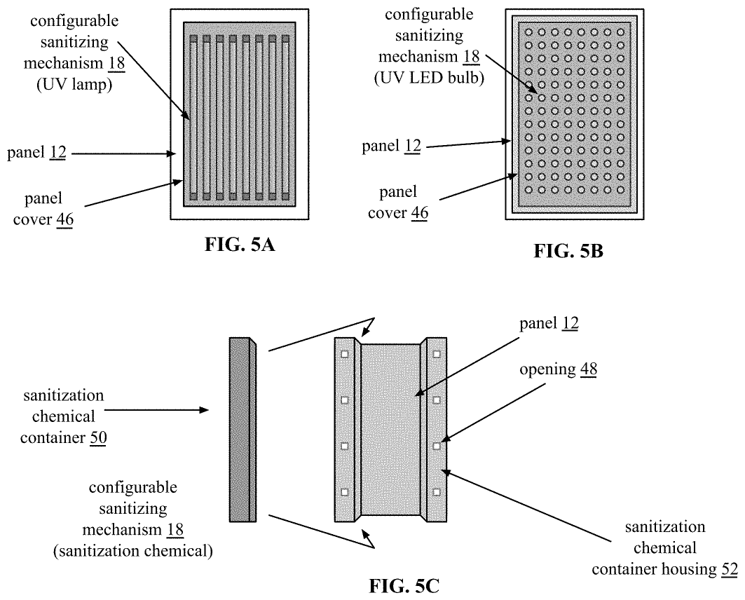

FIGS. 5A-5C are schematic block diagrams of examples of configurable sanitization mechanisms 18. FIG. 5A depicts an example of a configurable sanitizing mechanism 18 of an array of UV lamps embedded in a panel 12. The panel 12 may include a panel cover 46 having light filtering, scratch resistant, dust resistant, crack resistant, and/or other protective or light manipulating features.

FIG. 5B depicts an example of a configurable sanitizing mechanism 18 that is an array of UV LED bulbs embedded in a panel 12. The panel 12 may include a panel cover 46 having light filtering, scratch resistant, dust resistant, crack resistant, and/or other protective or light manipulating features. While multiple round UV LED bulbs are shown, other sizes and shapes (e.g., round, rectangular, etc.) are possible.

FIG. 5C depicts an example of a configurable sanitizing mechanism 18 that is a sanitization chemical. A panel 12 includes one or more sanitization chemical container housings 52 for one or more sanitization chemical containers 50. A sanitization chemical container 50 may be of a size and shape to fit into the sanitization chemical container housing 52. As shown in FIG. 5C, the sanitization chemical container 50 is a rectangular prism shape that fits into a rectangular prism shaped housing 52 but other shapes and sizes are possible. In another embodiment, the panel 12 is the sanitization chemical container housing 52. The sanitization chemical container 50 may be of the same or different material compared to the panel 12 and/or the sanitization chemical container housing 52. For example, the sanitization chemical container 50 is a plastic that is not eroded by the particular chemicals it contains, whereas the panel 12 is constructed of a lightweight metal material.

In this example, the panel 12 contains a plurality of openings 48 for dispensing the sanitization chemical. The sanitization chemical container 50 is connected to the panel 12 such that sanitization chemicals can be dispensed via the openings 48. For example, the sanitization chemical container is connected to a tubing connected to a power source and the sanitization chemical container housing 52 where the tubing controls the flow of a liquid sanitization chemical and allows sanitization chemicals to flow through the openings at a desired time (e.g., when triggered by a proximity sensor) and at a desired dose (e.g., as determined by the sanitization subject and controlled by the processing module).

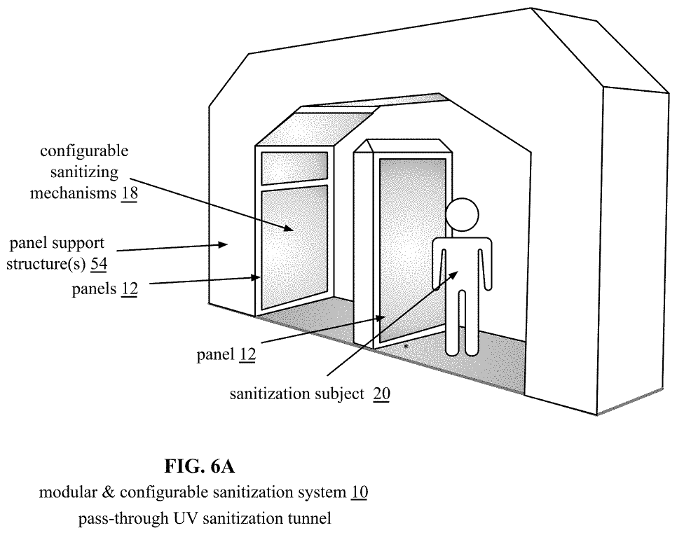

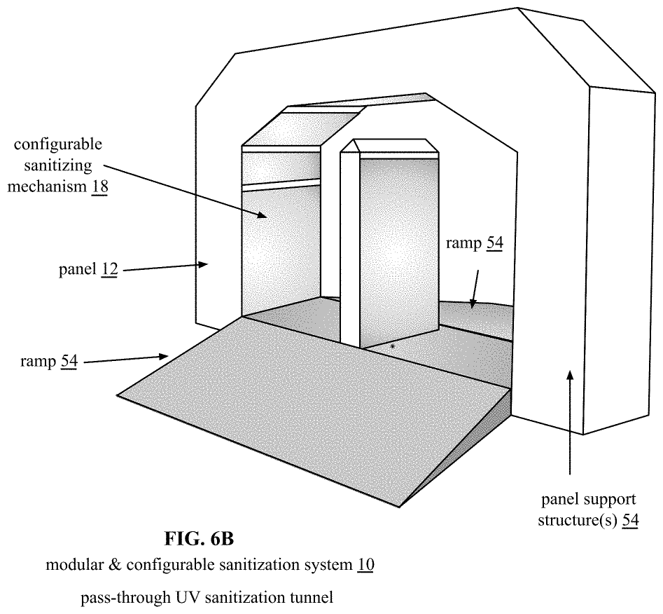

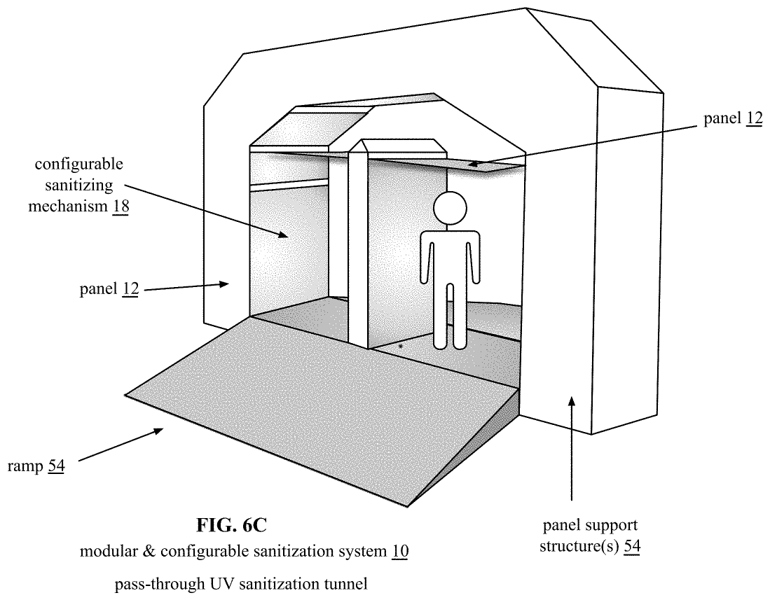

FIGS. 6A-6C are schematic block diagrams of an embodiment of a modular and configurable sanitization system 10 configured as a pass-through UV sanitization tunnel (also referred to herein as an “entrance tunnel,” “entrance UV tunnel,” and “tunnel”) that includes a plurality of panels 12, a plurality of configurable sanitization mechanisms 18, one or more modular panel support structures 54, one or more sanitization subjects 20, one or more power sources (e.g., battery, wiring for an external power source connection, etc.), and a processing module. The pass-through UV sanitization tunnel may include a control panel for receiving user inputs (e.g., on and off, settings, etc.) to the processing module and/or a display for displaying messages/and or instructions.

In FIGS. 6A-6C, the configurable sanitizing mechanisms 18 are UV lamp and/or bulb arrays and the sanitization subject 20 is anything that is able to pass through the tunnel such as a human being (as shown in FIG. 6A), objects the human being is carrying (e.g., a purse, briefcase, etc.), service animals, wheelchairs, carts, luggage, etc. The UV lamp arrays are embedded into the panels 12 forming one or more sides and partitions, a ceiling, and a floor of the entrance tunnel and supported structurally by one or more panel support structures 54 (e.g., building materials not containing sanitization mechanisms but used to connect and/or support one or more panel, and the overall shape of the intended panel configuration end use, etc.) in order to surround an individual and/or object with UV light when passing through the tunnel. As shown in FIG. 6A, the pass-through UV sanitization tunnel of this example includes a partition panel embedded with UV lamps on both sides to accommodate two sanitization subjects at a time.

The panels 12 are constructed of a suitable material for holding UV lamps and/or bulbs (e.g., metal, glass, plastic, etc.), may include one or more mechanisms for holding and/or affixing UV lamps and/or bulbs within and/or to the panels 12 (e.g., screw-in bulb base, light socket, adaptor, adhesive, attachment hardware, etc.), are of a size a shape in accordance with one or more portions of the tunnel (e.g., a side piece, floor piece, etc.), and are connected (e.g., with hardware such as screws, hardware-less joints that snap and/or fit snugly together, adhesive, etc.) into the shape of the tunnel.

The pass-through UV sanitization tunnel can be used and/or customized for a variety of public and private settings. For example, a pass-through UV sanitization tunnel can be set up at the entrance of a public destination that has multiple entries and/or exits in a given time period (e.g., a day, hour, etc.) such as a grocery store, restaurant, and/or medical facility. Due to its modular panel components, the UV entrance tunnel is easy to assemble for a temporary event such as a convention, trade show, or concert. As another example, a smaller scale UV entrance tunnel may be used at the consumer level such as within a person's home. Individuals exposed to contagions at work (e.g., healthcare workers, first responders, etc.), social gatherings, public spaces, etc., can pass through a UV entrance tunnel (e.g., set up in garage entry) prior to entering his or her home.

The UV lamps may be light-emitting diode (LED) lamps at a selectable wavelength, mercury-based lamps operating at low vapor pressure to emit a particular wavelength (e.g., similar to fluorescent lamps), pulsed-xenon lamps emitting UV light across the entire UVC spectrum, and/or a combination thereof. UV LED lamps have a selectable wavelength and more targeted, controllable light beams (e.g., traditional mercury lamps scatter light over a large area).

Because the UV light used here is intended for direct human exposure, far-UVC lamps having wavelengths between 207-222 nm may be used. Unlike conventional germicidal UVC (e.g., having wavelengths between 254 nm to 270 nm), far-UVC light inactivates bacteria and viruses without harming human tissue. Other germicidal, anti-microbial technology safe for human exposure may be implemented.

The UV lamp type and wattage is chosen based on the distance to target and desired dose of UV necessary for sanitization. For example, UV dose is generally calculated at 1 meter from a lamp. To ensure effectiveness, the UV dose is calculated based on UV intensity, distance to target, bulb and/or panel coating (e.g., shatter proof, scratch resistant, etc. can reduce UV output), end of lamp life, airflow, and exposure time. UV intensity is increased by introducing multiple UV lamps. For the UV entrance tunnel, a high dose of UV is necessary to disinfect people and/or objects in seconds as they enter the structure.

The floor panels (and possibly the panels on the other surfaces) are covered in a durable, transparent, anti-scratch, anti-dust material made to withstand high foot traffic such as treated glass, acrylic, polycarbonate, or other suitable material.

Various sensing technologies may be incorporated in the UV entrance tunnel such as scratch and/or dust detection (e.g., via infrared sensing). When a scratch and/or dust is detected, UV light is adjusted to compensate for the scratch and/or dust. For example, beam manipulation and light direction technology is used to direct light around the blocked area. As another example, affected bulbs are turned off to save power. As another example, a message is sent to a control panel notifying a user that cleaning and/or repair is necessary for proper function.

UV light only disinfects what is directly in its light path. Sensing and tracking technology may be used to better direct light onto the irregular contours of an individual's body. For example, as a person walks through, the person's shape, height, rate of walk, etc. are sensed and the bulbs are adjusted to direct light at the person in appropriate angles at certain times.

Different microorganisms require different amounts of time to inactivate/destroy. Therefore, to fully disinfect a person's exterior, the person may be instructed to pause within the structure for a certain amount of time. An individual may be asked (e.g., by a recorded voice or displayed message triggered by a proximity sensor) to stand in a certain position for a certain amount of time in order to ensure a desired dose and application of UV sanitization (e.g., with legs and arms spread out). For example, an individual may need to stand for approximately 20-30 seconds in the entrance tunnel.

As another example, the person may be instructed to turn to one side or another and/or to complete a full 360 degree turn within the UV entrance tunnel. Alternatively or additionally, automatic bulb adjustment, light filtering and beam direction can be used to direct beams of UV light to desired areas. Light filtering through different materials, lenses, etc., can also be used to adjust the intensity, frequency, and/or wavelength of the beams in order to produce a desired effect (e.g., specific intensities and wavelengths selected depending on the target, etc.).

The UV lamps may emit continuous light, pulsed light, or a combination thereof. Pulsed light may increase the life span of the UV lamps. The UV lamps may operate on a proximity sensor for power saving purposes such that the lamps are only activated when an individual is in close proximity.

The entrance tunnel may include counting, identification verification, tracking, camera, and/or facial recognition features for data collection and security purposes. For example, the entrance includes optical scanning technology (e.g., a barcode scanner) to scan an attendee's entrance badge and track who is currently at an event. As another example, a counting feature tracks the amount of people inside a facility at any one time. Counting may be performed based on touch and/or pressure sensing triggered when a person steps onto the floor of the UV entrance tunnel. Alternatively, counting is achieved through badge scanning, and/or camera/video tracking features of the UV entrance tunnel.

An enclosed UV sanitization unit may be present on one or more of the entrance or exit side of the UV tunnel for sterilizing personal items. The enclosed UV sterilization unit may contain panels embedded with UV lamps where the panels form the shape of a small to medium-sized box suitable to fit a jacket, suitcase, purse, gloves, wallet, and/or any other small personal items. The enclosed UV sanitization unit includes conventional germicidal UVC lamps or far-UVC lamps and operates on a timer. The enclosed UV sterilization unit may provide a higher dose of UV light to items that may not be fully disinfected during a quick walk through (e.g., a wallet is taken out of a pocket for disinfecting).

As an example, a person places a suitcase in the enclosed UV sterilization unit, shuts the top, enters the tunnel for disinfecting, and proceeds to the other side of the enclosed UV sterilization unit to retrieve disinfected items. When sterilized, the enclosed UV sterilization unit notifies the person to retrieve the disinfected items. If conventional UVC lamps are used, the UV sanitization unit includes a locking mechanism and an automatic shut off to prevent human exposure.

One or more additional configurable sanitization mechanisms may be included in one or more panels of the entrance tunnel such as sanitization chemical dispensers (e.g., a proximity sensor triggered, automatic hand sanitizer dispenser affixed to a panel of the entrance tunnel), air purification technology, etc.

FIG. 6B depicts another example of the modular and configurable sanitization system 10 as the pass-through UV sanitization tunnel that includes one or more entrance and exit ramps 54. The floor panels of the pass-through UV sanitization tunnel are likely raised to accommodate the UV lamps, a power source, etc., that are embedded in and/or affixed thereto. If necessary and/or desired, for safety (e.g., to prevent tripping), accessibility (e.g., for wheelchair access), and/or ease of use, a suitable ramp 54 can be added to the entrance and/or exit of the pass-through UV sanitization tunnel. The ramp 54 may be a traditional ramp of a suitable material and size and/or the ramp 54 may be a panel containing a configurable sanitizing mechanism.

FIG. 6C depicts another example of the modular and configurable sanitization system 10 as the pass-through UV sanitization tunnel that includes one or more ramps 54 and one or more additional panels 12 forming an awning out of the tunnel partition panel. The additional panels 12 may include light filtering and/or beam forming technology for directing, targeting, reducing, and/or distributing the UV light above a targeted individual. For example, a human being is more likely to look down and to the sides of the entrance tunnel upon entering rather than directly upwards. Therefore, safer wavelengths of UV light is used on the side and floor panels of the tunnel and the additional panels direct a higher wavelength over the top of the individual (where the higher wavelength is still safe for human tissue but may be less safe for direct human eye contact).

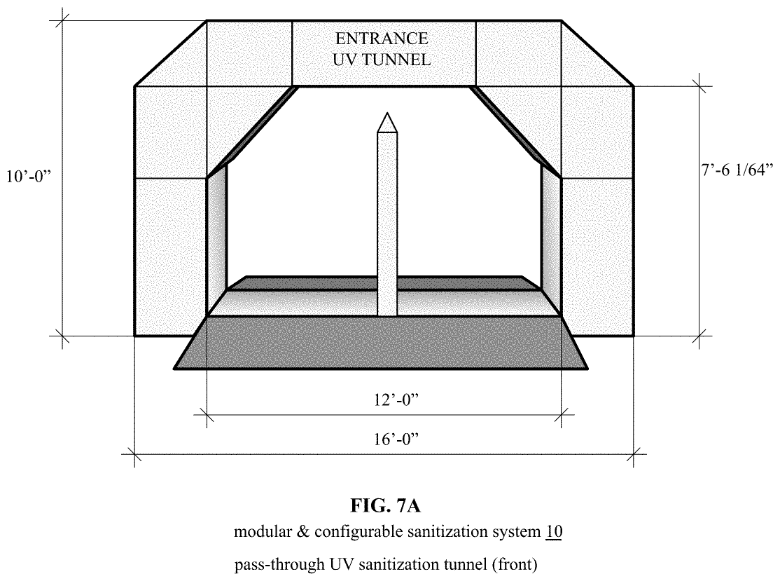

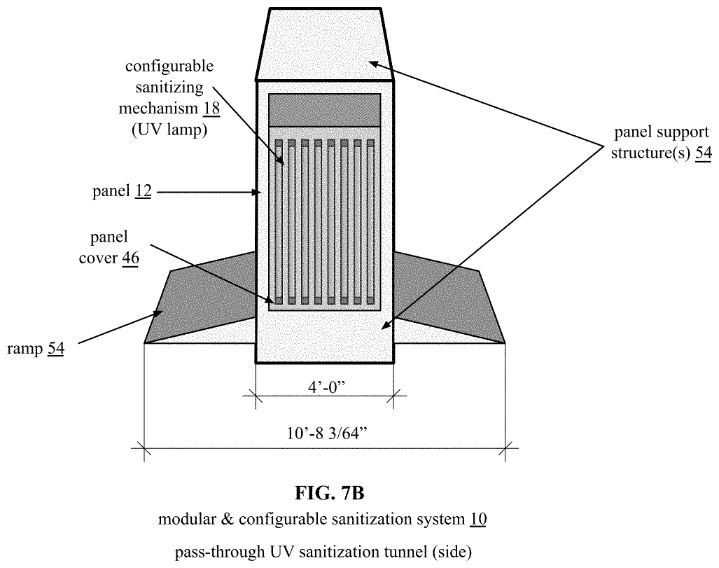

FIGS. 7A-7B are schematic block diagrams of embodiments of a modular and configurable sanitization system 10 configured as a pass-through UV sanitization tunnel. FIG. 7A shows a front view of an example pass-through UV sanitization tunnel and provides example dimensions of the sides, panel support structures, and ramps. Other dimensions and pass-through UV sanitization tunnel designs are possible.

FIG. 7B shows a side view of the example pass-through UV sanitization tunnel and provides example dimensions. The side panel support structure 54 shown in FIG. 7B is transparent to show the details of the panel on directed towards the side interior of the tunnel. In another embodiment the panel support structures 54 are hollow support structures such that the ceiling and sides are supported, and the panels can be seen from the exterior of the tunnel. In another embodiment, the panel support structures 54 are hollow support structures and include a transparent covering such that the panels can be seen from the exterior of the tunnel. The side panel 12 includes the configurable sanitizing mechanism 18 of an embedded array of UV lamps. The panel 12 may include a panel cover 46 having light filtering, scratch resistant, dust resistant, crack resistant, and/or other protective or light manipulating features. Other dimensions and pass-through UV sanitization tunnel designs are possible.

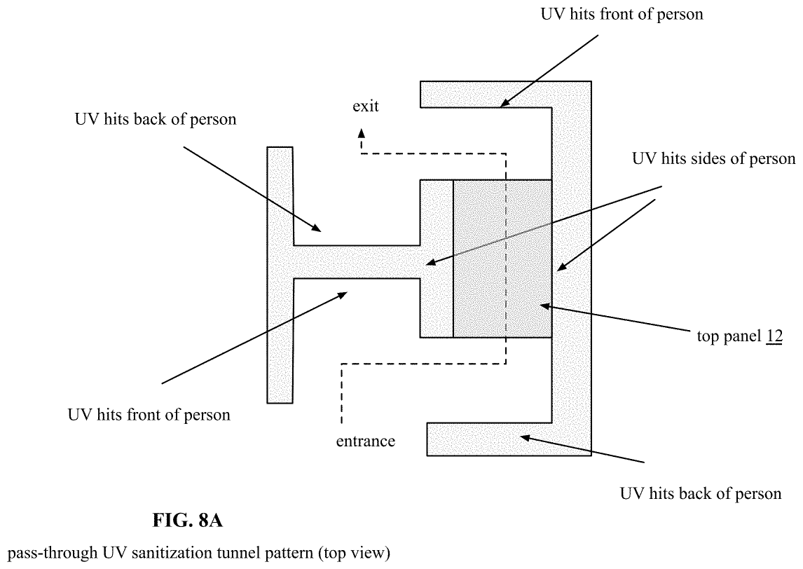

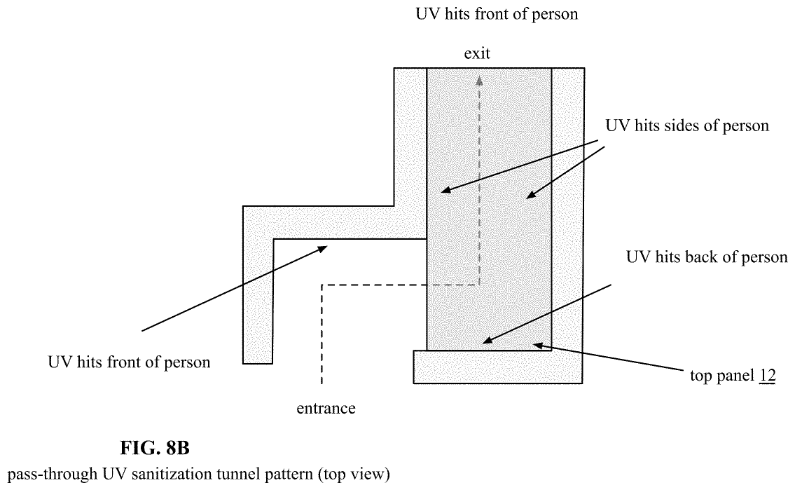

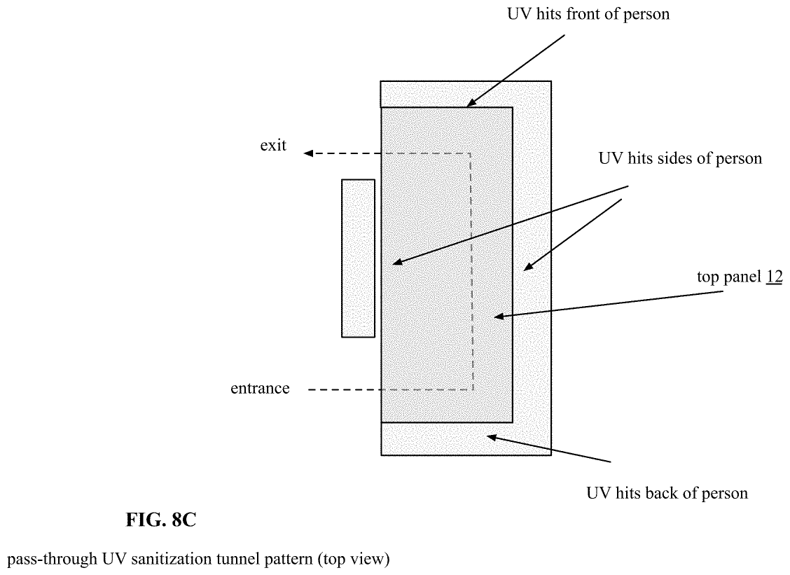

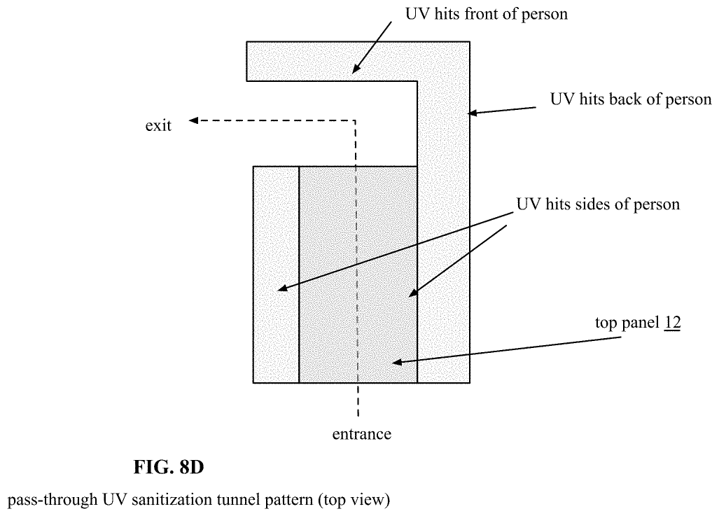

FIGS. 8A-8D are schematic block diagrams of examples of pass-through UV sanitization tunnel patterns. FIGS. 8A-8D are shown from a top view and includes panel walls embedded with far-UVC lamps similar to examples discussed above. At least a portion of the floor and the ceiling would require a UV lamp panel to sterilize bottom-up and top-down of an individual. FIG. 8A depicts a tunnel pattern to ensure that person is hit with UV from the back, front, and sides. As the person enters, he or she is hit with UV from the front and is forced to turn right into the tunnel. If the individual makes a quick right and avoids the first wall, the individual is still hit with UV from the front as he or she walks through the tunnel. Passing through the tunnel, the person is hit from the back, the sides, front, top panel, and a floor panel. As the person exits, the person is hit again from the back. The design works in a similar manner if entrance and exit are reversed. The design ensures the individual is thoroughly exposed to the UV light while never subjecting the individual to the confinement of a fully enclosed space. The tunnel pattern can be designed with a particular dimensions to ensure that at an average rate of egress and size of a sanitization subject, a particular dosage of UV is applied by the time it takes to pass through the tunnel.

FIG. 8B is a simplified version of FIG. 8A. In FIG. 8B, as the person enters, he or she is hit with UV from the front and is forced to turn right into the tunnel. If the individual makes a quick right and avoids the first wall, the individual is still hit with UV from the side wall of the tunnel while turning. Passing through the tunnel, the person is hit from the back, the sides, top panel, and a floor panel. As the person exits, the person is hit again from the back. The design works in a similar manner if entrance and exit are reversed.

In FIG. 8C, the individual is brought into the tunnel space through a left turn. Passing through the tunnel, the person is hit from the front, back, the sides, top panel, and a floor panel. The design works in a similar manner if entrance and exit are reversed.

In FIG. 8D, the individual is brought into the tunnel space immediately with no turn. Passing through the tunnel, the person is hit from the front, the sides, top panel, and a floor panel. As the person exits, he or she is forced to turn left which allows UV to hit the back of the person. The design works in a similar manner if entrance and exit are reversed except that the person turns initially and walks straight out.

Alternatively, or additionally, the UV entrance tunnel may include one or more moving UV lamp panels. For example, a UV lamp panel starts at a person's back and moves to the side, then the front, and the other side to ensure all sides of a person are sanitized.

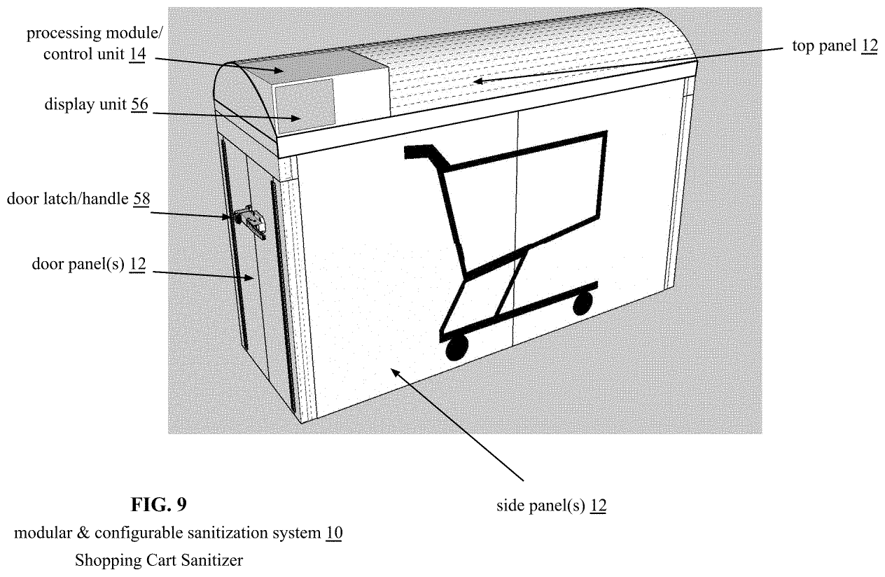

FIG. 9 is a schematic block diagram of an embodiment of a modular and configurable sanitization system 10 as a shopping cart sanitizer that includes a plurality of panels 12, a processing module/control unit 14, one or more door latches and/or handles 58, a display unit 56, and one or more power sources. The shopping cart sanitizer shown here is for sanitization of a single shopping cart. However, the shopping cart sanitizer can be designed to accommodate multiple shopping carts. The shopping cart sanitizer includes a plurality of modular panels such that it is easily taken down and reassembled. The interior panels of the structure can be embedded with conventional germicidal UVC lamp panels. In another embodiment, far-UVC lamps may be used throughout or just at the ends where human exposure is more likely.

The processing module/control unit 14 configures the UV lamps for a desired dosage and/or turns one or more lamps off as required. The display unit 56 is coupled to the processing module/control unit 14 and may include user input functionality to turn the shopping cart sanitizer on and off and display messages such as notifications of when lamps are on and when it is clear to open doors and remove the cart. The shopping cart sanitizer may be constructed with thermoforming plastic panels (e.g., pressure vacuum panels) over an internal steel frame. The top panel 12 may be powder coated for durability.

In an example of operation, a shopping cart is placed inside the structure at the entrance side, the doors are closed, the UVC lamps are illuminated for a period of time (e.g., 20 seconds), and a sanitized cart ready for use at the exit side of the structure. The cart can then be removed through the exit doors and placed in a “ready to use” cart line. The shopping cart sanitizer may include a conveyer belt mechanism to move the cart through the structure and out the other end.

In an example of use outside of a store, a dirty cart corral designated for un-sanitized shopping carts (e.g., designated by a red color) and a clean cart corrals designated for sanitized carts (e.g., designated by a green corral) indicate to customers where to leave a cart and where to pick up a cart. To prevent customers from operating the shopping cart sanitizer, the shopping cart sanitizer area can be fenced off and a shopping cart attendant (e.g., “cart wrangler”) moves carts from the dirty cart corrals (e.g., via a no touch device) in position for placement into the shopping cart sanitizer.

In embodiments where the shopping cart sanitizer is designed to house and sanitize multiple carts (i.e., a shopping cart sanitizing tunnel), the shopping cart sanitizer may be open on both ends (e.g., no doors are used). For example, used carts are placed into a designated end and sanitized carts come out the other side (e.g., manually based on pushing of carts through or via conveyor belt mechanics). If conventional germicidal UVC lamps are used, safety measures are required. For example, the conventional UVC lamps are used only in the center of the tunnel and far-UVC lamps (or no UV lamps) are used in areas closers to the open ends of the tunnel to reduce human exposure to harmful UVC. As another example, UVC lamps in the center of the tunnel provide a higher UV dose the lamps toward the open ends. As another example, the UV shopping cart sanitizer tunnel includes a proximity sensor such that lamps are only turned on when no people or animals are detected nearby.

In order to effectively sanitize each shopping cart, the carts need to be unstacked. Separators can be placed on the ends of shopping carts to ensure that the carts are not overlapped and are appropriately spaced for thorough sanitization of each cart. The UV shopping cart sanitizer tunnel may include a conveyor belt mechanism to move carts through the tunnel at a desired speed.

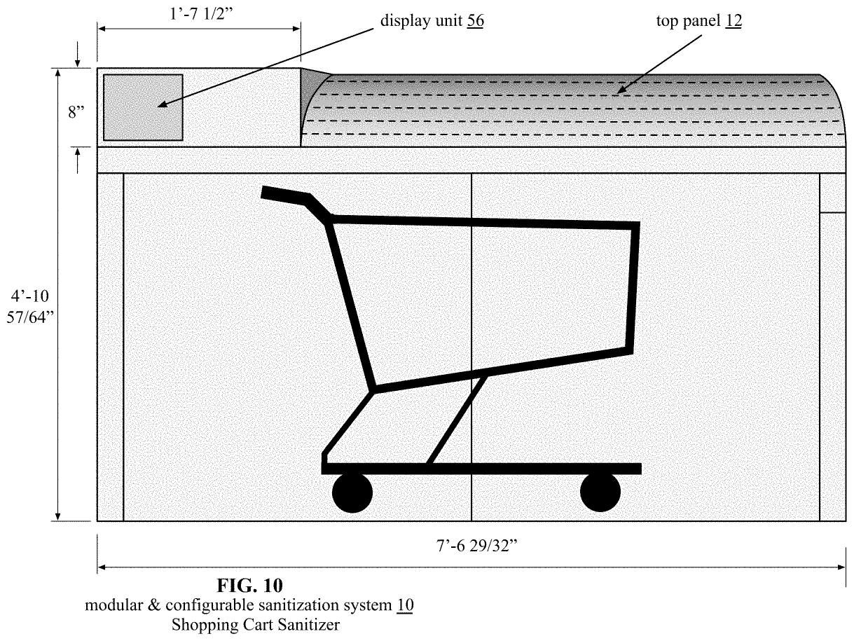

FIG. 10 is a schematic block diagram of a modular and configurable sanitization system 10 as a shopping cart sanitizer that includes a plurality of panels 12, a processing module/control unit 14, one or more door latches and/or handles 58, a display unit 56, and one or more power sources. The shopping cart sanitizer of FIG. 10 operates similarly to the shopping cart sanitizer of FIG. 9 except that it shows a different angle and includes example dimensions. Other dimensions, shapes, sizes, etc., are possible.

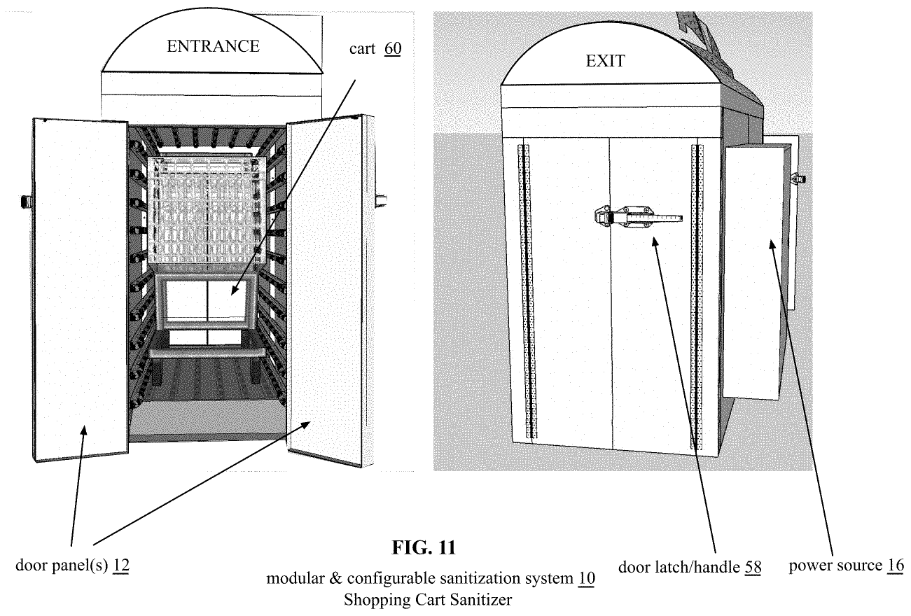

FIG. 11 is a schematic block diagram of an embodiment of a modular and configurable sanitization system 10 as a shopping cart sanitizer. FIG. 11 shows a cart 60 placed in the entrance side of the shopping cart sanitizer and a latched closed exit side of the shopping cart sanitizer. Both the entrance and exit sides of the structure may include locking mechanisms to ensure safety. The doors of the shopping cart sanitizer are shown here as two door panels 12 connected by a door latch/handle 58, however, the door may include one panel or more than two panels and the door latch/handle 58 may have a different placement than shown.

Alternatively, the shopping cart sanitizer may not include a door latch/handle 58 but a different mechanism for controlling entrance to the shopping cart sanitizer such as a manual button or motion/proximity sensor technology that opens, shuts, and/or locks the door(s) such that the doors close and lock while the UV light is on and unlock and open when the UV light is off and/or when the button and/or sensors are triggered. In FIG. 11, the door panels 12 are not embedded with UV lamps. The door panels 12 may include a layer of insulation material to further prevent exposure of UV rays emitted through the front and back of the unit and/or to protect the materials of the panels from degradation and/or exposure.

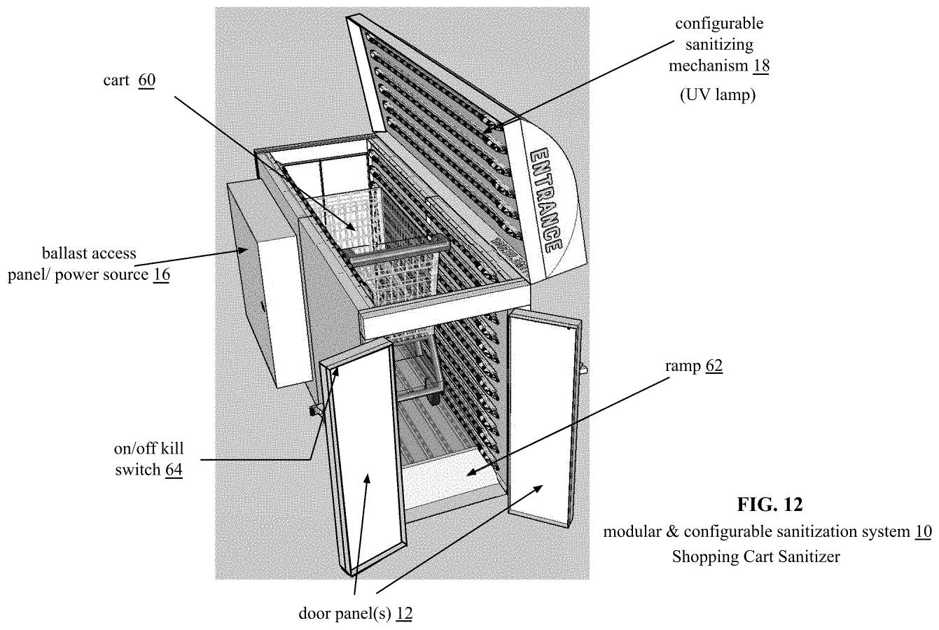

FIG. 12 is a schematic block diagram of another embodiment of a modular and configurable sanitization system 10 as a shopping cart sanitizer. FIG. 12 depicts an interior view of the shopping cart sanitizer of previous Figures that shows the configurable sanitizing mechanisms 18 (UV lamp arrays) embedded into the top, side, and floor panels of the shopping cart sanitizer, a ballast access panel/power source 16, an on/off kill switch 64, and a raised ramp 62.

The on/off switch 64 for the shopping cart sanitizer may be located in the control panel however an additional on/off kill switch 64 (e.g., a button) allows a user to shut down the shopping cart sanitizer at any time. For safety, the UV lamps may operate only when the doors are closed. Opening a door during sanitization activates a kill switch 64 to prevent exposure of UVC to human tissue. The ballast access panel/power source 16 regulates the current to the UV lamps and provides sufficient voltage to start the lamps.

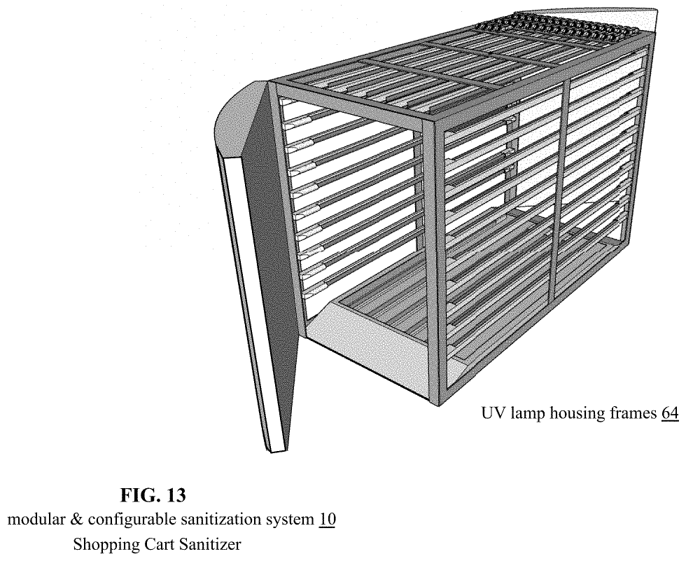

FIG. 13 is a schematic block diagram of another embodiment of a modular and configurable sanitization system 10 as a shopping cart sanitizer. FIG. 13 depicts the UV lamp housing frames 64 of the shopping cart sanitizer panels that hold the UV lamps. The UV lamp housing frames 64 in this example are constructed with steel or other suitable material for UV lamp array housing.

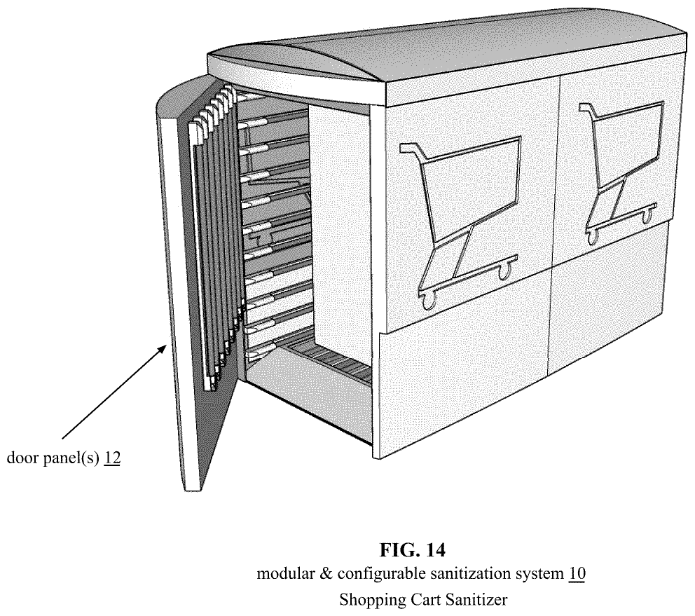

FIG. 14 is a schematic block diagram of another embodiment of a modular and configurable sanitization system 10 as a shopping cart sanitizer. The shopping cart sanitizer of FIG. 14 operates similarly to the shopping cart sanitizer of FIGS. 9-13 except that the shopping cart sanitizer of FIG. 14 includes a different exterior body style and includes a full door panel 12 (not a split door as shown in FIG. 11) that is embedded with UV lamps. As shown, a shopping cart is completed surrounded by UV light when placed inside the shopping cart sanitizer.

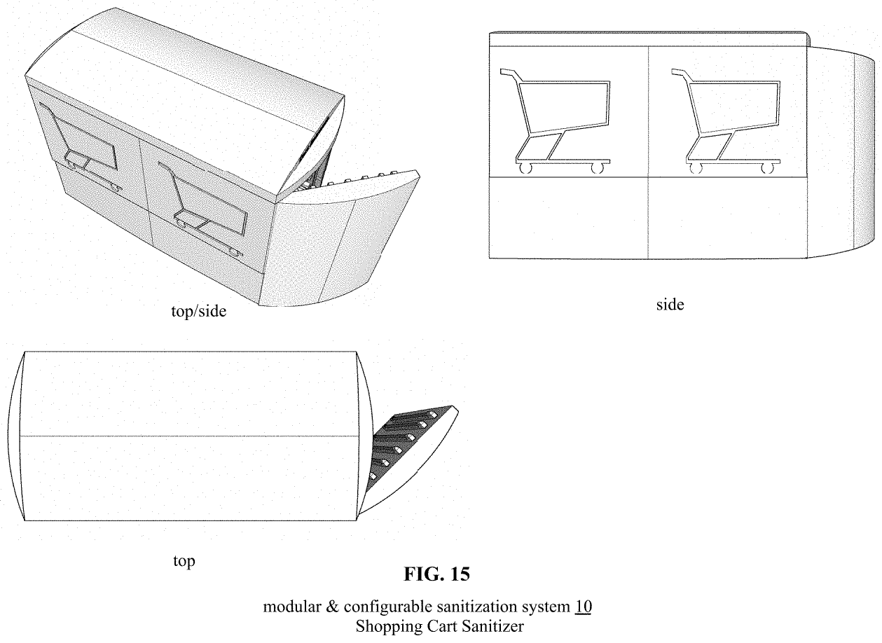

FIG. 15 is a schematic block diagram of another embodiment of a modular and configurable sanitization system 10 as a shopping cart sanitizer. FIG. 15 depicts an example top/side view, a side view, and a top view of the exterior body style of the shopping cart sanitizer of FIG. 14.

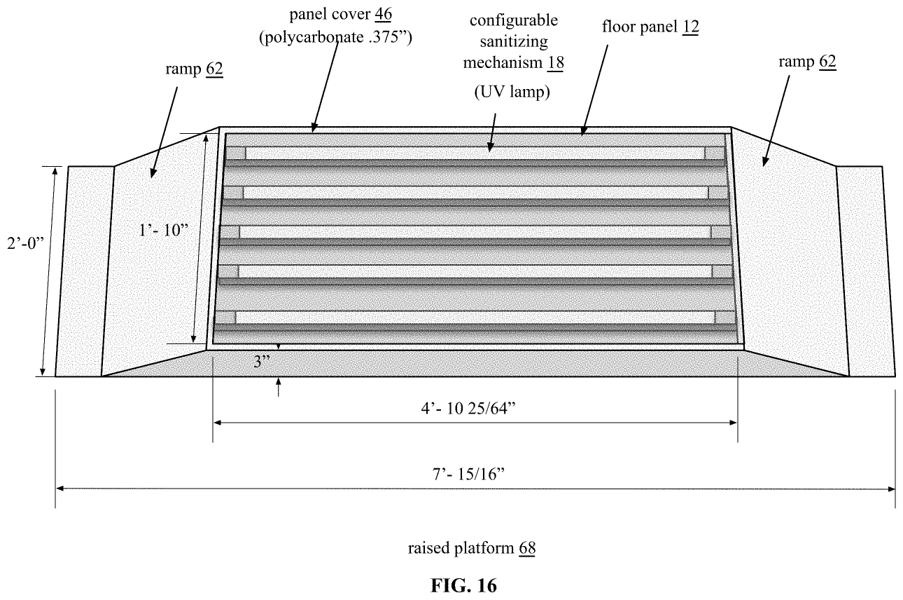

FIG. 16 is a schematic block diagram of an example of a raised platform 68 of a shopping cart sanitizer. The raised platform 68 includes a floor panel 12 embedded with configurable sanitizing mechanisms 18 (UV lamps), a panel cover 46, and ramps 62 on either side to allow for a shopping cart to roll up onto the raised platform for full exposure to the UV light. The panel cover 46 is a transparent, durable material such as polycarbonate. FIG. 16 includes example dimensions of the raised platform 68 but other dimensions and designs are possible.

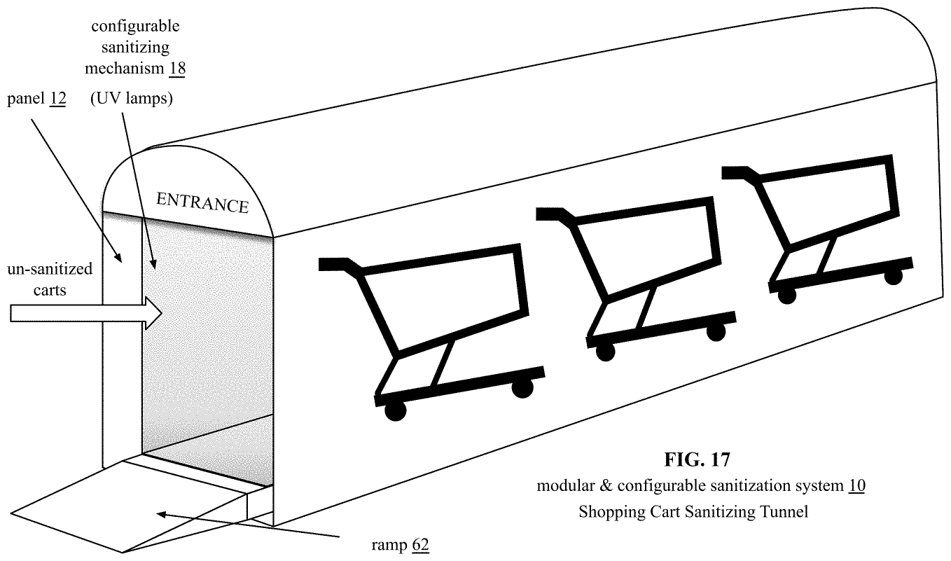

FIG. 17 is a schematic block diagram of an embodiment of a modular and configurable sanitization system 10 as a shopping cart sanitizing tunnel. The shopping cart sanitizing tunnel operates similarly to the shopping cart sanitizer of previous Figures except that shopping cart sanitizing tunnel may not include doors and is long enough to sanitize multiple shopping carts at a time. The shopping cart sanitizing tunnel includes a plurality of panels 12, a plurality of configurable sanitizing mechanisms 18 (e.g., UV lamp arrays), and a ramp 62.

The shopping cart sanitizing tunnel may include a conveyer belt mechanism to move carts through the structure and out the other end. Users or designated cart wranglers place un-sanitized shopping carts up the ramp 62 and into an open entrance of the shopping cart sanitizing tunnel. In an alternative embodiment, the shopping cart sanitizing tunnel includes a conveyor belt that lifts the carts up and onto the ramp and into the shopping cart sanitizing tunnel.

The shopping cart sanitizing tunnel may be open on both ends (e.g., no doors are used) or include one or more doors similar to the examples previously discussed. When no doors are included, for example, used (i.e., un-sanitized) carts are placed into a designated end (e.g., “entrance”) and sanitized carts come out the other side (e.g., by manually pushing the carts through, via a conveyor belt mechanics, etc.). If conventional germicidal UVC lamps are used, safety measures are required. For example, the conventional UVC lamps are used only in the center of the tunnel and far-UVC lamps (or no UV lamps) are used in areas closer to the open ends of the tunnel to reduce human exposure to harmful UVC. As another example, UVC lamps in the center of the tunnel provide a higher UV dose the lamps toward the open ends. As another example, the UV shopping cart sanitizer tunnel includes a proximity sensor such that lamps are only turned on when no people or animals are detected nearby.

In order to effectively sanitize each shopping cart, the carts need to be unstacked. Separators can be placed on the ends of shopping carts to ensure that the carts are not overlapped and are appropriately spaced for thorough sanitization of each cart. The UV shopping cart sanitizer tunnel may include a conveyor belt mechanism to move carts through the tunnel at a desired speed.

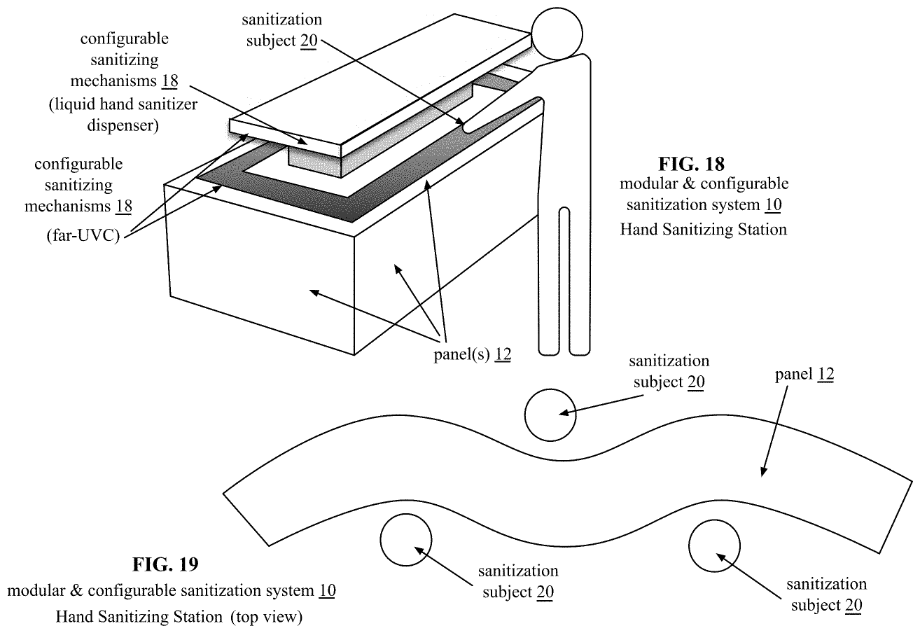

FIG. 18 is a schematic block diagram of an embodiment of modular and configurable sanitization system 10 as a hand sanitizing station. The hand sanitizing station includes modular panels 12 for easy teardown and reassembly. The hand sanitizing station includes a plurality of configurable sanitizing mechanisms 18 such as liquid hand sanitizer dispensers and/or embedded human safe far-UVC lamps (or other safe germicidal technology).

The sanitization subject 20 is a human being's hands and possibly forearm area. In this example, the hand sanitizing station is configured as a size and shape suitable for multiple people to stand around the station, however; it could be designed for a single user and/or designed at a smaller/different scale (e.g., for children, handicap accessible, etc.). The human safe far-UVC lamps may be embedded in panels in places above and below where a hand would be placed. The liquid hand sanitizer dispensers are placed such that when presence of a hand is detected via motion, proximity sensing, etc., an appropriate dosage of hand sanitizer hits the sanitization subject 20.

FIG. 19 is a schematic block diagram of another embodiment of a modular and configurable sanitization system 10 as a hand sanitizing station. The hand sanitizing station of FIG. 19 operates similarly to the hand sanitizing station of FIG. 18 except that the hand sanitizing station of FIG. 19 shows a top view and has a curved structure to direct users to particular areas of the hand sanitizing station based on where dispensers are located, privacy, spacing, social distancing, space reduction, for a streamlined and pleasing appearance, etc.

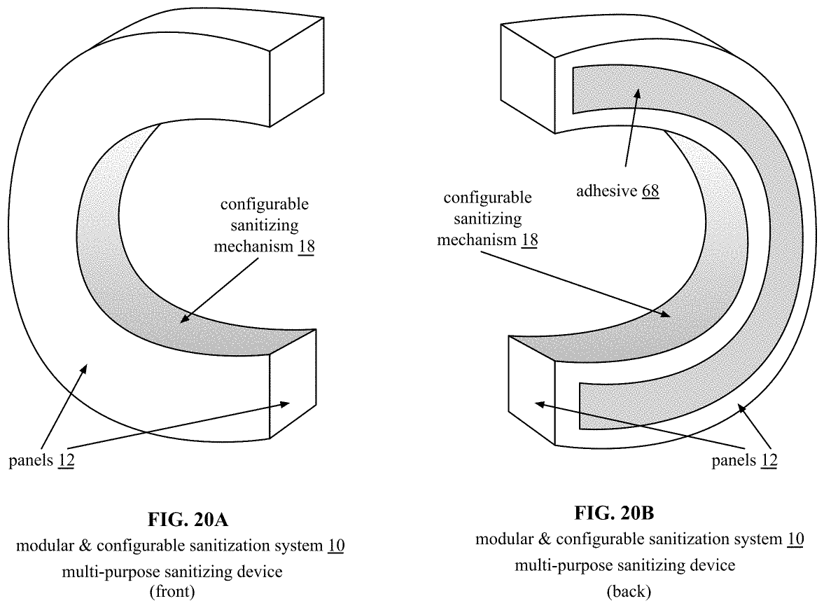

FIGS. 20A-20B are schematic block diagrams of an embodiment of a modular and configurable sanitization system 10 as a multi-purpose sanitizing device. FIG. 20A shows a front view of the multi-purpose sanitizing device that includes a plurality of panels 12 and a configurable sanitizing mechanism 18. The multi-purpose sanitizing device further includes and/or is connectable to a processing module, one or more power sources, and/or one or more sensing devices. The multi-purpose sanitizing device has a half circle shape here, but any type of size and shape of multi-purpose sanitizing device is possible.

FIG. 20B depicts a back view of the multi-purpose sanitizing device that includes a plurality of panels 12, a configurable sanitizing mechanism 18, and adhesive 68. The adhesive 68 may be a replaceable adhesive strip that adheres the multi-purpose sanitizing device to a variety of surfaces and can be taken off with minimal to no damage. Alternatively, the adhesive may be a more permanent adhesive (e.g., glue, hardware, a combination thereof, etc.) such that the multi-purpose sanitizing device is adhered to a particular location as a permanent or semi-permanent fixture.

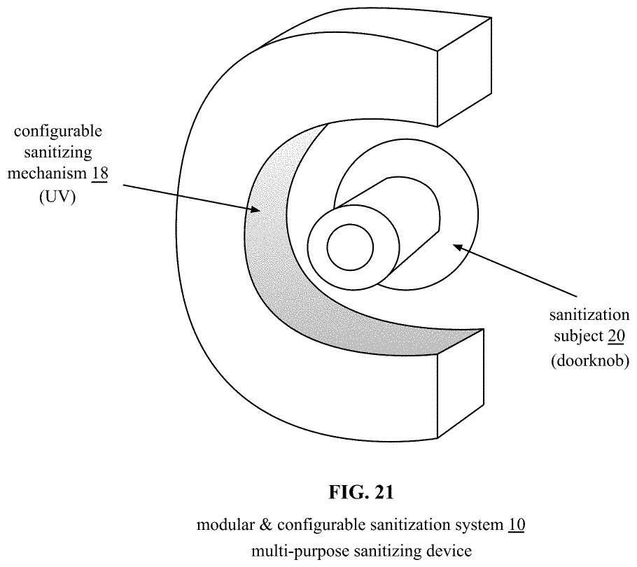

FIG. 21 is a schematic block diagram of an embodiment of a modular and configurable sanitization system 10 as a multi-purpose sanitizing device. In this example, the multi-purpose sanitizing device is configured to fit around a sanitization subject 20 of a doorknob (and/or door handle) for continuous or intermittent sanitization. For example, when the configurable sanitizing mechanism includes conventional germicidal UVC light, a proximity sensor is included to detect human presence in the disinfecting areas. When an individual is in close proximity, the UVC light turns off.

When human-safe UVC light is used (e.g., far-UVC), the UVC light may be kept on continuously, may be sensor triggered to sanitize after a touch is detected, and/or to turn off after a certain amount of time for power conservation. The multi-purpose sanitizing device is constructed using appropriately sized and shaped UV lamps/bulbs for the sanitization subject area and the power and intensity required.

For example, an array of small UV LED bulbs is used for small panels and disinfection of items close to the panel, and larger UV LED lamps are used for larger panels and disinfecting larger areas. Multi-purpose sanitizing devices can be embedded on and/or adhered to high touch areas such as shopping cart handles, retail shelving units, cash registers, etc. Multi-purpose sanitizing devices using human safe UV light can be installed in and/or adhered to ceiling panels, floors, and/or wall panels for continuous disinfection of high traffic public areas (grocery stores, malls, hospitals, etc.).

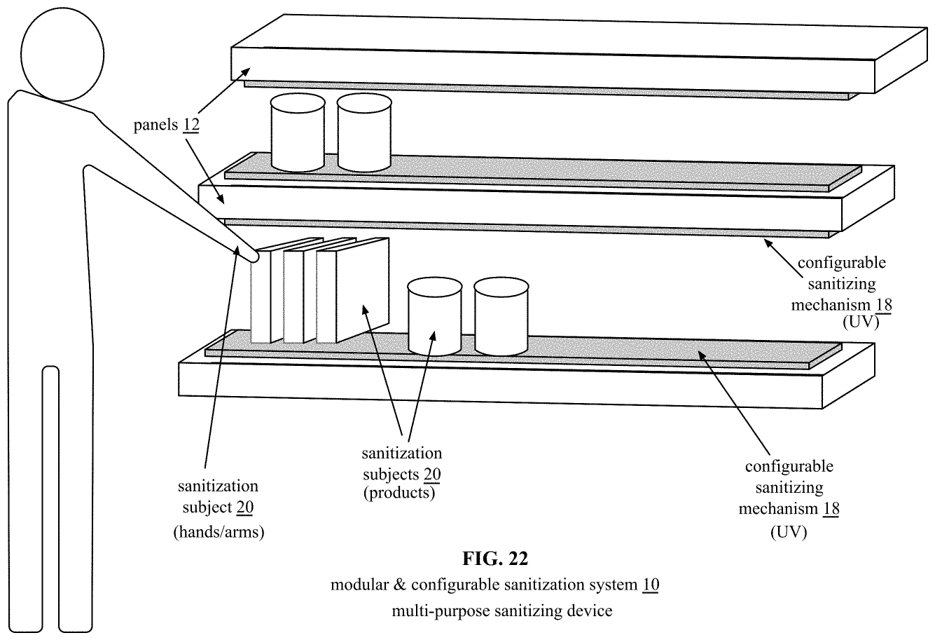

FIG. 22 is a schematic block diagram of another embodiment of a modular and configurable sanitization system 10 as a multi-purpose sanitizing device. In this example, the multi-purpose sanitizing devices are constructed for adherence to a surface of a shelf of a shelving unit for continuous or intermittent sterilization of sanitization subjects 20 (e.g., items/products on the shelves, and/or objects that come in contact with the shelves such as human arms and hands). For example, the panels 12 of the multi-purpose sanitizing device are embedded with a configurable sanitizing mechanism 18 such as UV lamps, are rectangular in shape, and are adhered to the top and bottom surfaces of the shelves using an adhesive means (e.g., removable adhesive strip, glue, hardware, and/or a combination thereof).

When conventional germicidal UVC light is used, a proximity sensor is included to detect human presence in the disinfecting areas. When an individual is in close proximity, the UVC light turns off. When human-safe UVC light is used (e.g., far-UVC), the UVC light may be kept on continuously, may be sensor triggered to sanitize after a touch is detected, and/or to turn off after a certain amount of time for power conservation. In this example, when an individual is in close proximity, the panels may switch from conventional germicidal UVC light to human-safe UVC light. The configurable sanitizing mechanism 18 is constructed using appropriately sized and shaped UV lamps/bulbs for the size of the area and the power and intensity required.

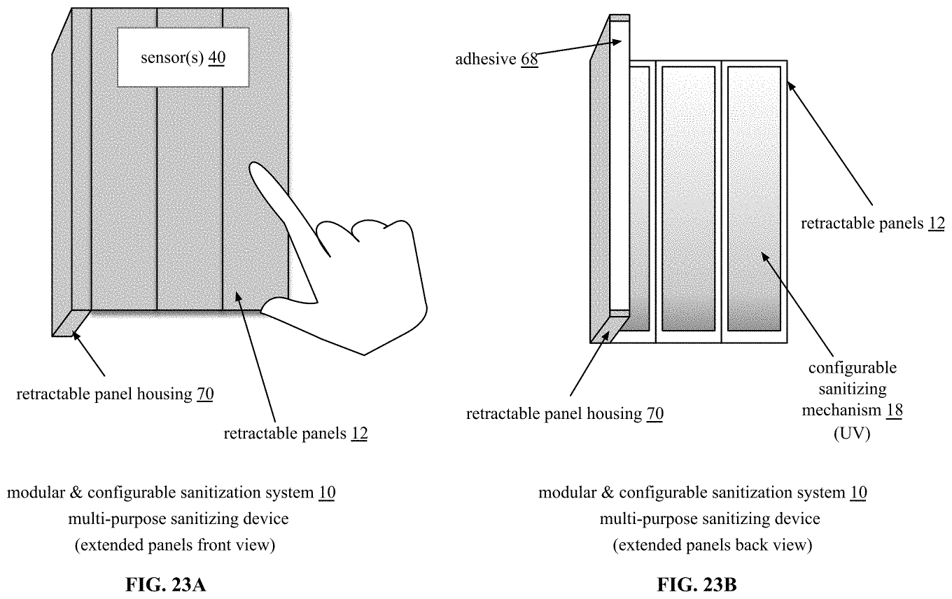

FIGS. 23A-23B are schematic block diagram of embodiments of a modular and configurable sanitization system 10 as a multi-purpose sanitizing device. FIG. 23A shows a front view of the multi-purpose sanitizing device that includes a plurality of retractable panels 12, a retractable panel housing 70, and one or more sensors 40. The plurality of retractable panels 12 of the multi-purpose sanitizing device include one or more configurable sanitizing mechanisms 18 for targeting a sanitization subject located under the extended panels 12.

The retractable panels 12 are mechanically configurable via automatic retraction technology (e.g., a panel includes a motor, a power source, and the retractable panel housing allowing the panel to roll/unroll or fold/unfold onto the housing (e.g., a motor powered tube housing) and/or automatic collapsing technology (e.g., the panel contains a motor, a power source, and has nested sections that fit into one another to allow for an adjustable length, height, etc.) such that panels can be automatically added and/or subtracted to the system and/or the shape of an a panel can be altered. In this example, the retractable panel housing 70 includes space for the retractable panel storage as well as a small motor and/or power source for automatic retraction.

The one or more configurable sanitizing mechanisms 18 may include conventional germicidal UVC light bulbs for continually or intermittently sanitizing a non-living sanitization subject (e.g., an elevator button, etc.).

When the sensor 40 (e.g., a proximity sensor, motion sensor, etc.) is triggered (e.g., the sanitization subject is an elevator button, and a person comes in close contact with the button) the UVC light turn off and the retractable panels 12 retract and store into the retractable panel housing 70 to expose the sanitization subject. The multi-purpose sanitizing device further includes and/or is connectable to a processing module, one or more power sources, and/or one or more other sensing devices. The multi-purpose sanitizing device has a rectangular shape here, but any type of size and shape of multi-purpose sanitizing device is possible.

FIG. 23B depicts a back view of the multi-purpose sanitizing device that includes a plurality of retractable panels 12, a retractable panel housing 70, and adhesive 68. From the back view, the configurable sanitizing mechanisms 18 are visible. The adhesive 68 may be a replaceable adhesive strip that adheres the multi-purpose sanitizing device to a variety of surfaces and can be taken off with minimal to no damage, or the adhesive may be a more permanent adhesive (e.g., glue, hardware, a combination thereof, etc.) such that the multi-purpose sanitizing device is adhered to a particular location as a permanent or semi-permanent fixture.

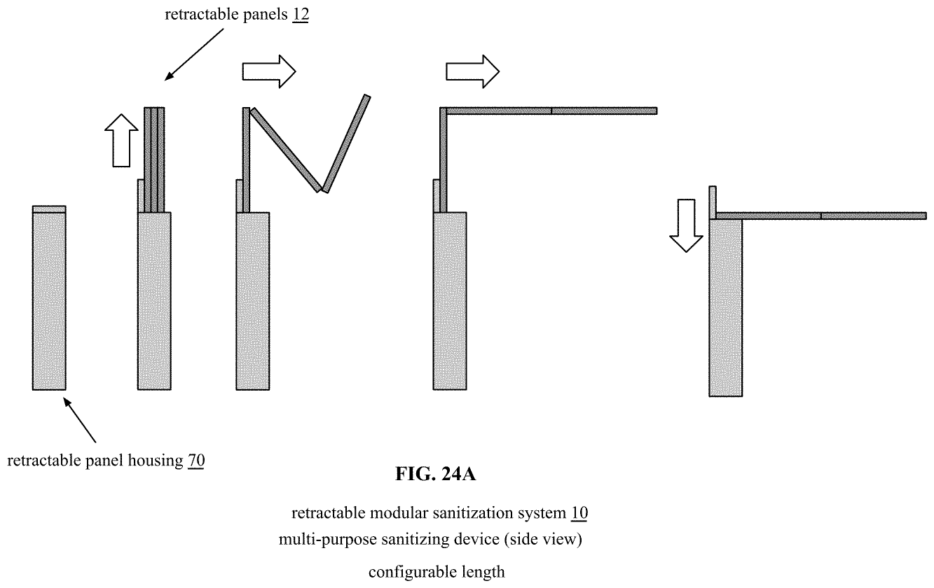

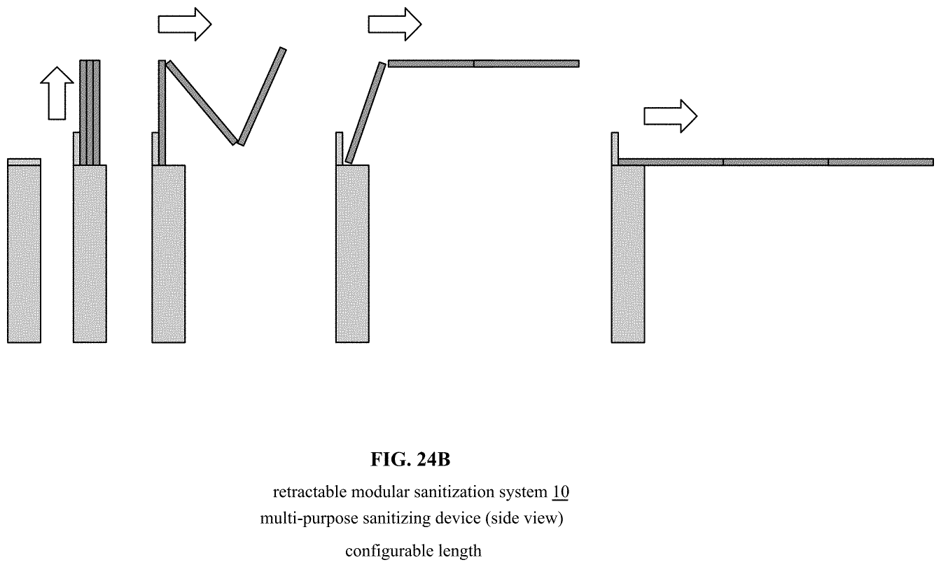

FIGS. 24A-24B are schematic block diagrams of another embodiment of a modular and configurable sanitization system 10 as a multi-purpose sanitizing device that includes a plurality of retractable panels 12, a retractable panel housing 70, and one or more sensors 40. The multi-purpose sanitizing device of FIGS. 24A-24B is similar to the multi-purpose sanitizing device of FIGS. 23A-23B expect that FIGS. 24A-24B depict a side view to illustrate a configurable length, panel retraction method.

For example, in FIG. 24A, at a first stage, the retractable panels 12 are housed in the retractable panel housing 70. At a second stage (e.g., when the multi-purpose sanitizing device determines sanitization is required (e.g., a human is no longer triggering a sensor)), the retractable panel housing 70 opens and the retractable panels 12 extend out from the retractable panel housing 70. In this example, three rectangular shaped retractable panels are depicted, however; different shapes and amounts of retractable panels are possible.

At a third stage, the retractable panels 12 begin to unfold, where the retractable panels 12 are connected to one another via flexible joints. At a fourth stage, two retractable panels 12 are unfolded and extended. In this example, the multi-purpose sanitizing device is configured to sanitize a sanitization subject of a particular size that does not require the full length of all the retractable panels 12. Therefore, at a fifth stage, the remaining retractable panels 12 retracts back down into the retractable panel housing 70 while the unfolded retractable panels 12 remain unfolded and extended for sanitization.

FIG. 24B is similar to the example of FIG. 24A except that the third retractable panel 12 is unfolded and extended such that the full length of the retractable panels 12 is extended for sanitization.

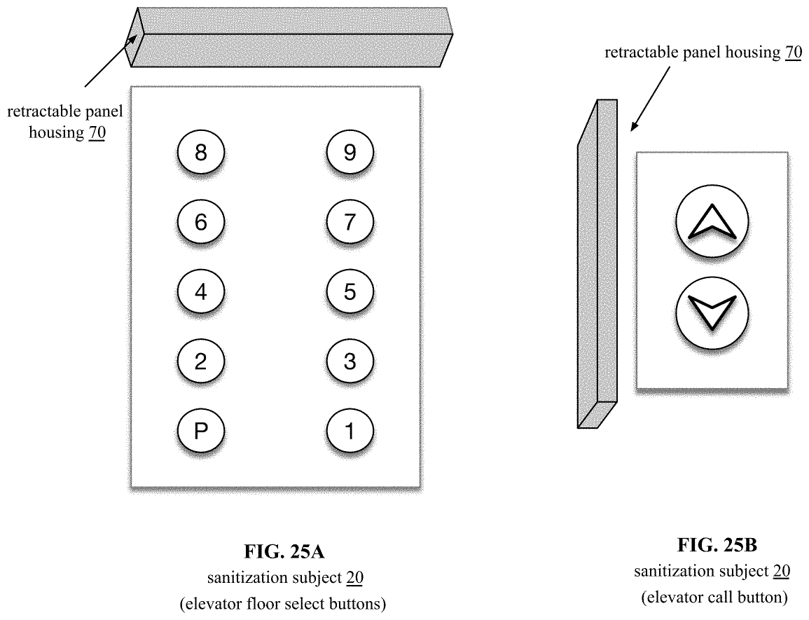

FIGS. 25A-25B are schematic block diagrams of embodiments of sanitization subjects 20 of a multi-purpose sanitizing device having retractable panels and one or more sensing devices. In FIG. 25A, the sanitization subject 20 is a set of elevator floor select buttons. The multi-purpose sanitizing device may be adhered above (as shown), below, or to one side of the sanitization subject 20 depending on the length of the extended retractable panels, the type of adhesion, location requirements, etc. As shown in FIG. 25A, the retractable panels are retracted and housed in the retractable panel housing 70 such that the sanitization subject 20 is exposed and available for use. When a person and/or touch is no longer sensed by one or more sensing devices of the multi-purpose sanitizing device, the retracted panels are operable to unfold and cover the sanitization subject 20 for sanitization.

In FIG. 25B, the sanitization subject 20 is an elevator call button. While the elevator call button is smaller than the set of elevator floor select buttons, it is possible for the same model of multi-purpose sanitizing device to be adhered above, below, or to one side of the sanitization subject 20 (as shown) where a size adjustment is made (e.g., not all of the retractable panels need to extend to cover the sanitization subject 20). As shown in FIG. 25B, the retractable panels are retracted and housed in the retractable panel housing 70 such that the sanitization subject 20 is exposed and available for use. When a person and/or touch is no longer sensed by one or more sensing devices of the multi-purpose sanitizing device, the retracted panels are operable to unfold and cover the sanitization subject 20 for sanitization.

It is noted that terminologies as may be used herein such as bit stream, stream, signal sequence, etc. (or their equivalents) have been used interchangeably to describe digital information whose content corresponds to any of a number of desired types (e.g., data, video, speech, text, graphics, audio, etc. any of which may generally be referred to as ‘data’).

As may be used herein, the terms “substantially” and “approximately” provides an industry-accepted tolerance for its corresponding term and/or relativity between items. For some industries, an industry-accepted tolerance is less than one percent and, for other industries, the industry-accepted tolerance is 10 percent or more. Other examples of industry-accepted tolerance range from less than one percent to fifty percent. Industry-accepted tolerances correspond to, but are not limited to, component values, integrated circuit process variations, temperature variations, rise and fall times, thermal noise, dimensions, signaling errors, dropped packets, temperatures, pressures, material compositions, and/or performance metrics. Within an industry, tolerance variances of accepted tolerances may be more or less than a percentage level (e.g., dimension tolerance of less than +/−1%). Some relativity between items may range from a difference of less than a percentage level to a few percent. Other relativity between items may range from a difference of a few percent to magnitude of differences.

As may also be used herein, the term(s) “configured to”, “operably coupled to”, “coupled to”, and/or “coupling” includes direct coupling between items and/or indirect coupling between items via an intervening item (e.g., an item includes, but is not limited to, a component, an element, a circuit, and/or a module) where, for an example of indirect coupling, the intervening item does not modify the information of a signal but may adjust its current level, voltage level, and/or power level. As may further be used herein, inferred coupling (i.e., where one element is coupled to another element by inference) includes direct and indirect coupling between two items in the same manner as “coupled to”.

As may even further be used herein, the term “configured to”, “operable to”, “coupled to”, or “operably coupled to” indicates that an item includes one or more of power connections, input(s), output(s), etc., to perform, when activated, one or more its corresponding functions and may further include inferred coupling to one or more other items. As may still further be used herein, the term “associated with”, includes direct and/or indirect coupling of separate items and/or one item being embedded within another item.

As may be used herein, the term “compares favorably”, indicates that a comparison between two or more items, signals, etc., provides a desired relationship. For example, when the desired relationship is that signal 1 has a greater magnitude than signal 2, a favorable comparison may be achieved when the magnitude of signal 1 is greater than that of signal 2 or when the magnitude of signal 2 is less than that of signal 1. As may be used herein, the term “compares unfavorably”, indicates that a comparison between two or more items, signals, etc., fails to provide the desired relationship.

As may be used herein, one or more claims may include, in a specific form of this generic form, the phrase “at least one of a, b, and c” or of this generic form “at least one of a, b, or c”, with more or less elements than “a”, “b”, and “c”. In either phrasing, the phrases are to be interpreted identically. In particular, “at least one of a, b, and c” is equivalent to “at least one of a, b, or c” and shall mean a, b, and/or c. As an example, it means: “a” only, “b” only, “c” only, “a” and “b”, “a” and “c”, “b” and “c”, and/or “a”, “b”, and “c”.

As may also be used herein, the terms “processing module”, “processing circuit”, “processor”, “processing circuitry”, and/or “processing unit” may be a single processing device or a plurality of processing devices. Such a processing device may be a microprocessor, micro-controller, digital signal processor, microcomputer, central processing unit, field programmable gate array, programmable logic device, state machine, logic circuitry, analog circuitry, digital circuitry, and/or any device that manipulates signals (analog and/or digital) based on hard coding of the circuitry and/or operational instructions. The processing module, module, processing circuit, processing circuitry, and/or processing unit may be, or further include, memory and/or an integrated memory element, which may be a single memory device, a plurality of memory devices, and/or embedded circuitry of another processing module, module, processing circuit, processing circuitry, and/or processing unit. Such a memory device may be a read-only memory, random access memory, volatile memory, non-volatile memory, static memory, dynamic memory, flash memory, cache memory, and/or any device that stores digital information. Note that if the processing module, module, processing circuit, processing circuitry, and/or processing unit includes more than one processing device, the processing devices may be centrally located (e.g., directly coupled together via a wired and/or wireless bus structure) or may be distributedly located (e.g., cloud computing via indirect coupling via a local area network and/or a wide area network). Further note that if the processing module, module, processing circuit, processing circuitry and/or processing unit implements one or more of its functions via a state machine, analog circuitry, digital circuitry, and/or logic circuitry, the memory and/or memory element storing the corresponding operational instructions may be embedded within, or external to, the circuitry comprising the state machine, analog circuitry, digital circuitry, and/or logic circuitry. Still further note that, the memory element may store, and the processing module, module, processing circuit, processing circuitry and/or processing unit executes, hard coded and/or operational instructions corresponding to at least some of the steps and/or functions illustrated in one or more of the Figures. Such a memory device or memory element can be included in an article of manufacture.

One or more embodiments have been described above with the aid of method steps illustrating the performance of specified functions and relationships thereof. The boundaries and sequence of these functional building blocks and method steps have been arbitrarily defined herein for convenience of description. Alternate boundaries and sequences can be defined so long as the specified functions and relationships are appropriately performed. Any such alternate boundaries or sequences are thus within the scope and spirit of the claims. Further, the boundaries of these functional building blocks have been arbitrarily defined for convenience of description. Alternate boundaries could be defined as long as the certain significant functions are appropriately performed. Similarly, flow diagram blocks may also have been arbitrarily defined herein to illustrate certain significant functionality.

To the extent used, the flow diagram block boundaries and sequence could have been defined otherwise and still perform the certain significant functionality. Such alternate definitions of both functional building blocks and flow diagram blocks and sequences are thus within the scope and spirit of the claims. One of average skill in the art will also recognize that the functional building blocks, and other illustrative blocks, modules and components herein, can be implemented as illustrated or by discrete components, application specific integrated circuits, processors executing appropriate software and the like or any combination thereof.

In addition, a flow diagram may include a “start” and/or “continue” indication. The “start” and “continue” indications reflect that the steps presented can optionally be incorporated in or otherwise used in conjunction with one or more other routines. In addition, a flow diagram may include an “end” and/or “continue” indication. The “end” and/or “continue” indications reflect that the steps presented can end as described and shown or optionally be incorporated in or otherwise used in conjunction with one or more other routines. In this context, “start” indicates the beginning of the first step presented and may be preceded by other activities not specifically shown. Further, the “continue” indication reflects that the steps presented may be performed multiple times and/or may be succeeded by other activities not specifically shown. Further, while a flow diagram indicates a particular ordering of steps, other orderings are likewise possible provided that the principles of causality are maintained.

The one or more embodiments are used herein to illustrate one or more aspects, one or more features, one or more concepts, and/or one or more examples. A physical embodiment of an apparatus, an article of manufacture, a machine, and/or of a process may include one or more of the aspects, features, concepts, examples, etc. described with reference to one or more of the embodiments discussed herein. Further, from figure to figure, the embodiments may incorporate the same or similarly named functions, steps, modules, etc. that may use the same or different reference numbers and, as such, the functions, steps, modules, etc. may be the same or similar functions, steps, modules, etc. or different ones.

Unless specifically stated to the contra, signals to, from, and/or between elements in a figure of any of the figures presented herein may be analog or digital, continuous time or discrete time, and single-ended or differential. For instance, if a signal path is shown as a single-ended path, it also represents a differential signal path. Similarly, if a signal path is shown as a differential path, it also represents a single-ended signal path. While one or more particular architectures are described herein, other architectures can likewise be implemented that use one or more data buses not expressly shown, direct connectivity between elements, and/or indirect coupling between other elements as recognized by one of average skill in the art.

The term “module” is used in the description of one or more of the embodiments. A module implements one or more functions via a device such as a processor or other processing device or other hardware that may include or operate in association with a memory that stores operational instructions. A module may operate independently and/or in conjunction with software and/or firmware. As also used herein, a module may contain one or more sub-modules, each of which may be one or more modules.

As may further be used herein, a computer readable memory includes one or more memory elements. A memory element may be a separate memory device, multiple memory devices, or a set of memory locations within a memory device. Such a memory device may be a read-only memory, random access memory, volatile memory, non-volatile memory, static memory, dynamic memory, flash memory, cache memory, a quantum register or other quantum memory and/or any other device that stores data in a non-transitory manner. Furthermore, the memory device may be in a form of a solid-state memory, a hard drive memory or other disk storage, cloud memory, thumb drive, server memory, computing device memory, and/or other non-transitory medium for storing data. The storage of data includes temporary storage (i.e., data is lost when power is removed from the memory element) and/or persistent storage (i.e., data is retained when power is removed from the memory element). As used herein, a transitory medium shall mean one or more of: (a) a wired or wireless medium for the transportation of data as a signal from one computing device to another computing device for temporary storage or persistent storage; (b) a wired or wireless medium for the transportation of data as a signal within a computing device from one element of the computing device to another element of the computing device for temporary storage or persistent storage; (c) a wired or wireless medium for the transportation of data as a signal from one computing device to another computing device for processing the data by the other computing device; and (d) a wired or wireless medium for the transportation of data as a signal within a computing device from one element of the computing device to another element of the computing device for processing the data by the other element of the computing device. As may be used herein, a non-transitory computer readable memory is substantially equivalent to a computer readable memory. A non-transitory computer readable memory can also be referred to as a non-transitory computer readable storage medium.

While particular combinations of various functions and features of the one or more embodiments have been expressly described herein, other combinations of these features and functions are likewise possible. The present disclosure is not limited by the particular examples disclosed herein and expressly incorporates these other combinations.

What is claimed is:

1. A modular and configurable sanitization system comprises:
one or more panels, wherein a panel of the one or more panels includes at least one configurable sanitizing mechanism; and
a processing module operably coupled to the one or more panels, wherein the processing module is operable to:
obtain configuration data pertaining to one or more of: the one or more panels, one or more configurable sanitizing mechanisms of the one or more panels, and one or more sanitization subjects;
determine one or more sanitization protocols based on the configuration data;
generate one or more sanitizing mechanism configuration instructions to configure the one or more configurable sanitizing mechanisms based on the one or more sanitization protocols and in accordance with the one or more sanitization subjects;
generate one or more panel configuration instructions to configure the one or more panels based on the one or more sanitization protocols and in accordance with the one or more sanitization subjects; and
configure the one or more configurable sanitizing mechanisms and the one or more panels based on the one or more sanitizing mechanism configuration instructions and the one or more panel configuration instructions.

2. The modular and configurable sanitization system of claim 1 further comprises:
one or more sensing devices coupled to one or more of: the processing module and the one or more panels.

3. The modular and configurable sanitization system of claim 1, wherein the processing module is operable to obtain the configuration data by one or more of:
receiving one or more data inputs;
performing a query and response process with the one or more of: the one or more panels, the one or more configurable sanitizing mechanisms, the one or more sanitization subjects, and one or more power sources of the modular and configurable sanitization system;
performing a data analysis process, wherein the data analysis process is based on one or more of: stored data, historical data, the one or more data inputs, and a data model; and
performing a lookup process.

4. The modular and configurable sanitization system of claim 1, wherein the configuration data includes one or more of:
characteristics of the one or more sanitization subjects;
sanitization requirements of the one or more sanitization subjects;
sanitization preferences of the one or more sanitization subjects;
a required panel configuration of the one or more panels;
a current panel configuration of the one or more panels;
a desired panel configuration of the one or more panels;
characteristics of the one or more configurable sanitizing mechanisms;
configuration capabilities of the one or more configurable sanitizing mechanisms;
power requirements pertaining to one or more of: the one or more sanitization subjects, the one or more panels, and the one or more configurable sanitizing mechanisms; and
default settings pertaining to one or more of: the one or more sanitization subjects, the one or more panels, and the one or more configurable sanitizing mechanisms.

5. The modular and configurable sanitization system of claim 1, wherein the sanitization protocols includes one or more of:
one or more sanitization methods;
one or more amount of sanitization;
a time period of sanitization;
one or more safety protocols; and
one or more sanitization areas.

6. The modular and configurable sanitization system of claim 1, wherein the one or more configurable sanitizing mechanisms include at least one of:
ultraviolet (UV) light application;
dispensing of sanitization chemicals;
air purification technology; and
water purification technology.

7. The modular and configurable sanitization system of claim 1, wherein the one or more sanitization subjects include one of:
at least a portion of a living being;
a surface;
an object;
water; and
air.

8. The modular and configurable sanitization system of claim 1, wherein the processing module is further operable to:
send the one or more sanitizing mechanism configuration instructions to the one or more configurable sanitizing mechanisms via an interface means.

9. The modular and configurable sanitization system of claim 1, wherein the processing module is further operable to:
send the one or more panel configuration instructions to the one or more panels via an interface means.

10. A method comprises:
obtaining, by a processing module of a modular and configurable sanitization system, configuration data pertaining to one or more of: one or more panels of the modular and configurable sanitization system, one or more configurable sanitizing mechanisms of the one or more panels, and one or more sanitization subjects of the modular and configurable sanitization system;
determining, by the processing module, one or more sanitization protocols based on the configuration data;
generating, by the processing module, one or more sanitizing mechanism configuration instructions to configure the one or more configurable sanitizing mechanisms based on the one or more sanitization protocols and in accordance with the one or more sanitization subjects;
generating, by the processing module, one or more panel configuration instructions to configure the one or more panels based on the one or more sanitization protocols and in accordance with the one or more sanitization subjects; and
configuring, by the processing module, the one or more configurable sanitizing mechanisms and the one or more panels based on the one or more sanitizing mechanism configuration instructions and the one or more panel configuration instructions.

11. The method of claim 10 further comprises:
obtaining, by the processing module, sensed data via one or more sensing device of the modular and configurable sanitization system; and
updating, by the processing module, the configuration data with the sensed data.

12. The method of claim 10, wherein the obtaining the configuration data comprises one or more of:
receiving, by the processing module, one or more data inputs;
performing, by the processing module, a query and response process with the one or more of: the one or more panels, the one or more configurable sanitizing mechanisms, the one or more sanitization subjects, and one or more power sources of the modular and configurable sanitization system;
performing, by the processing module, a data analysis process, wherein the data analysis process is based on one or more of: stored data, historical data, the one or more data inputs, and a data model; and
performing, by the processing module, a lookup.

13. The method of claim 10, wherein the configuration data includes one or more of:
characteristics of the one or more sanitization subjects;
sanitization requirements of the one or more sanitization subjects;
sanitization preferences of the one or more sanitization subjects;
a required panel configuration of the one or more panels;
a current panel configuration of the one or more panels;
a desired panel configuration of the one or more panels;
characteristics of the one or more configurable sanitizing mechanisms;
configuration capabilities of the one or more configurable sanitizing mechanisms;
power requirements pertaining to one or more of: the one or more sanitization subjects, the one or more panels, and the one or more configurable sanitizing mechanisms; and
default settings pertaining to one or more of: the one or more sanitization subjects, the one or more panels, and the one or more configurable sanitizing mechanisms.

14. The method of claim 10, wherein the sanitization protocols includes one or more of:
one or more sanitization methods;
one or more amount of sanitization;
a time period of sanitization;
one or more safety protocols; and
one or more sanitization areas.

15. The method of claim 10, wherein the one or more configurable sanitizing mechanisms include at least one of:
ultraviolet (UV) light application;
dispensing of sanitization chemicals;
air purification technology; and
water purification technology.

16. The method of claim 10, wherein the one or more sanitization subjects include one of:
at least a portion of a living being;
a surface;
an object;
water; and
air.

17. The method of claim 10 further comprises:
sending, by the processing module, the one or more sanitizing mechanism configuration instructions to the one or more configurable sanitizing mechanisms via an interface means.

18. The method of claim 10 further comprises:
sending, by the processing module, the one or more panel configuration instructions to the one or more panels via an interface means.
